(19) United States
(12) Patent Application Publication
(10) Pub. No.: US 2020/0246318 A1
(43) Pub. Date: Aug. 6, 2020

(54) METHODS OF TREATING PAIN WITH A THIAZOLINE ANTI-HYPERALGESIC

(71) Applicant: CERSCI THERAPEUTICS, INC., Dallas, TX (US)

(72) Inventor: SCOTT L. DAX, LANDENBERG, PA (US)

(21) Appl. No.: 16/720,819

(22) Filed: Dec. 19, 2019

Related U.S. Application Data
(60) Provisional application No. 62/800,232, filed on Feb. 1, 2019.

(51) Int. Cl. A61K 31/426; A61P 3/10; A61K 9/20; A61K 9/02; A61K 9/00; A61K 9/48; A61P 29/00

(52) U.S. Cl. CPC A61K 31/426; A61P 3/10; A61K 9/20; A61P 29/00; A61K 9/0056; A61K 9/48; A61K 9/02

(57) ABSTRACT

Methods of treating diabetic neuropathic pain and post-surgical pain are provided. The methods include administering to an individual a therapeutically effective amount of a compound of Formula I (Compound 1). The method can be used to treat diabetic neuropathy arising from any type of nerve damage, and can also be used to treat post-surgical pain arising from any surgical procedure without the side effects associated with widely used analgesics such as opioids. Compound 1 can be formulated into many suitable dosage forms, including oral dosage forms such as tablets.

### CROSS-REFERENCE TO RELATED APPLICATION

This application claims priority to U.S. Provisional Patent Application Ser. No. 62/800,232 entitled “METHODS OF TREATING PAIN WITH A THIAZOLIDINE ANTI-HYPERALGESIC,” filed Feb. 1, 2019, the disclosure of which is incorporated herein by reference in its entirety.

### BACKGROUND

Pain is defined as an unpleasant sensory and emotional experience. Pain, however, can be informative and useful. For example, nociceptive pain is often indicative of injury (e.g., tissue damage), and such pain typically evokes escape or protective behaviors in animals or in a human, in order to remove itself, or protect itself, from further exposure to the insult. However, inflammation, cellular and neuronal damage and other processes resulting from injury or disease can lead to states of chronic pathological pain. Hyperalgesia is a condition in which enhanced sensitivity to noxious stimuli is present, and thus the perception of pain is exaggerated. Allodynia is a condition in which normally non-noxious stimuli become painful. Persistent or chronic pain, manifested as hyperalgesia and/or allodynia, remains challenging to treat. Many patients do not respond to existing therapeutics, or have their pain poorly managed (i.e., inadequate relief), or experience relief of an inadequate duration.

Endogenous reactive species produced by injury, irritant and disease are key drivers of pain as can be demonstrated in animal models of hyperalgesia and allodynia. Reactive oxygen species (ROS) and reactive nitrogen species (RNS) include free radicals such as superoxide and hydroxyl radical as well as the powerful oxidants peroxynitrite (OONO⁻), and (hydrogen) peroxide (H₂O₂). Both peroxynitrite (PN) and hydrogen peroxide, generated in the periphery after injury, contribute to changes in excitability in sensory afferents.

Peroxynitrite has been implicated in the development of opiate-induced antinociceptive (pain) tolerance (tachyphylaxis) (Muscoli et al., 2007, J Clin Invest 117:3530-3539). Peroxynitrite results from the diffusion-controlled reaction of superoxide (O₂⁻) and nitric oxide (NO). Unlike other endogenously produced reactive species/oxidants, peroxynitrite is not managed by enzymatic control. Peroxynitrite formation is facile, unleashing its powerful oxidative properties essentially unchecked, causing downstream effects that can cause pain.

In contrast, superoxide is formed from the action of NADPH oxidases and xanthine oxidase, and nitric oxide is produced by nitric oxide synthases (NOS). Hydrogen peroxide is formed from superoxide and the action of superoxide dismutase. During cellular stress (e.g., inflammation, nerve injury, ischemia), the action of these enzymatic systems can cause nitric oxide, superoxide and peroxide levels to increase significantly, which can lead to neuronal damage, hyperalgesia and allodynia. Concomitant increases in nitric oxide and superoxide can lead to greatly increased localized increases in peroxynitrite, which is capable of nitrating tyrosine residues within proteins, cross-linking cysteine residues and disrupting glutathione-disulfide homeostasis. Collectively, these effects lead to neuronal sensitization and pain, including neuropathic pain.

Diabetes is a leading cause of neuropathy. Approximately 50% of diabetic patients will develop peripheral neuropathy which manifests as burning, excruciating, stabbing or intractable types of pain. The currently available therapeutics are palliative, effective in only a portion of patients in providing symptomatic relief, and are not disease-modifying (diabetes). More troubling, even patients who initially experience relief from a given therapeutic usually revert to a painful state over time. Anticonvulsants such as pregabalin, gabapentin and lamotrigine and older tricyclic antidepressants (TCA) such as carbamazepine can be effective but are prone to produce CNS-associated adverse effects (e.g., sedation, cognitive deficits). Antidepressants belonging to the norepinephrine- and/or serotonin-reuptake inhibitors (SNRIs) class such as duloxetine are useful alternatives in some patients. The use of opioids and non-steroidal anti-inflammatory drugs (NSAIDs) is commonplace but not preferable due to abuse potential, withdrawal, tolerance leading to dose-escalation, constipation, nausea, vomiting and respiratory depression well-known to occur with opioid therapy and gastrointestinal ulceration and nephrotoxicity associated with NSAID usage. Lastly, topical agents (capsaicin, topical nitrates and topical TCAs) and local anesthetics have been used with mixed results.

Collectively, the treatment of painful diabetic neuropathy remains poorly managed as evident by Numbers-Needed-to-Treat values which range from 5 to 6 for the mostly widely used drugs (NEURONTIN®, LYRICA®, CYMBALTA®) (Treatment of Painful Diabetic Neuropathy-, Ther. Adv. Chronic Dis. 2015, 6 (1) 15 (S Javed).

Post-operative pain is another source of pain that needs better treatment options than exist today. Post-operative pain is frequently the result of surgery, but other treatments such as, for example, management of acute pain following burns or non-surgical trauma can also result in severe pain. Post-operative pain management is important to reduce or eliminate pain and discomfort so that the surgical patient can begin ambulating as soon as possible, which speeds recovery.

The surgical site has a marked effect on the degree of post-operative pain. In general, surgery on the thorax and upper abdomen are more painful than surgery on the lower abdomen, which in turn is more painful than peripheral surgery on the limbs. In particular, thoracic surgery or upper abdominal surgery can produce extensive changes in pulmonary function, a decrease in abdominal muscle tone and a related decrease in diaphragmatic function. Decreased function in the diaphragm can produce an inability to cough and clear mucus, which can lead to lung collapse and/or pneumonia. Persistent pain can reduce physical activity and mobility and lead to increased risk of deep vein thrombosis and pulmonary embolisms. These problems are unpleasant or even life-threatening and often result in extended hospital stays. Patients that have moderate to severe post-surgical pain frequently require pain control at least in the first 3 days after trauma or surgery, and often as much as 2 to 3 weeks post-surgery.

There is a need in the medical and patient communities for a new class of therapeutic agents that can relieve a wide range of pain, including, but not limited to painful diabetic neuropathy and post-surgical pain. The methods and compounds described herein address this pressing need.

### SUMMARY OF THE INVENTION

In various embodiments, a method of treating diabetic neuropathy is provided. The method comprises administering a therapeutically effective amount of a composition comprising a compound of Formula I:

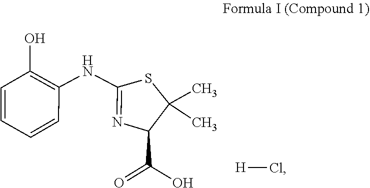 [SMILES: CC1(C)SC(Nc2ccccc2O)=N[C@@H]1C(=O)O ; Cl]

to an individual having diabetic neuropathy.

In various embodiments, a method of treating post-surgical pain is provided. The method comprises administering a therapeutically effective amount of a composition comprising a compound of Formula I:

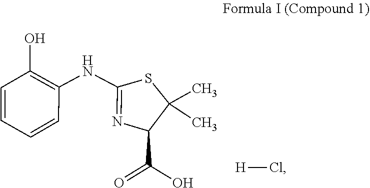 [SMILES: CC1(C)SC(Nc2ccccc2O)=N[C@@H]1C(=O)O ; Cl]

to an individual having post-surgical pain.

In various embodiments, a method of making a compound of Formula I (Compound 1) is provided. The method comprises reacting an amine with a structure of

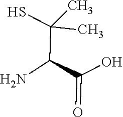

with

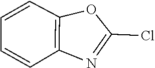

in the presence of a base and a first solvent to form an intermediate product of Formula II:

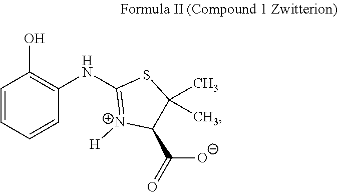 [SMILES: CC1(C)SC(Nc2ccccc2O)=[NH+][C@@H]1C(=O)[O-]]

and contacting the intermediate product with an acid and a second solvent to form the compound of Formula I.

In various embodiments, a kit comprising a composition comprising a compound of Formula I,

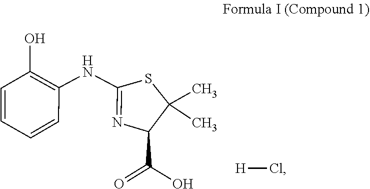 [SMILES: CC1(C)SC(Nc2ccccc2O)=N[C@@H]1C(=O)O ; Cl]

an applicator, and instructional material for use thereof is provided. The instructional material includes instructions for treating diabetic neuropathy or post-surgical pain.

### BRIEF DESCRIPTION OF THE FIGURES

The drawings illustrate generally, by way of example, but not by way of limitation, various embodiments of the present invention.

FIG. 1 is an X-ray crystal structure of (R)-2-(2-hydroxyphenylamino)-5,5-dimethyl-4,5-dihydrothiazole-4-carboxylic acid mono-hydrochloride (Compound 1), in accordance with various embodiments.

FIG. 2 is an infrared (IR) spectrum of Compound 1, in accordance with various embodiments.

FIG. 3 is a ¹H-NMR (nuclear magnetic resonance) spectrum of Compound 1, in accordance with various embodiments.

FIG. 4 is a ¹³C-NMR spectrum of Compound 1, in accordance with various embodiments.

FIG. 5 is an experimental (bottom trace) and calculated XRPD (X-ray powder diffraction) trace (top trace) for Compound 1, in accordance with various embodiments.

FIG. 6 is a GVS isotherm plot for Compound 1, in accordance with various embodiments.

FIG. 7 is a combined DSC/TGA trace for Compound 1, in accordance with various embodiments.

FIG. 8 is a listing of structures of impurities potentially formed during the manufacture of Compound 1, in accordance with various embodiments.

FIG. 9 is a listing of structures of impurities potentially formed during the manufacture of Compound 1 from Compound 1 Zwitterion, in accordance with various embodiments.

FIG. 10 illustrates non-limiting effects of Compound 1 on hyperalgesia in a rodent incisional model, in accordance with various embodiments.

FIG. 11 illustrates non-limiting efficacy of Compound 1 in an incision-induced hyperalgesia model compared to Celecoxib and morphine, in accordance with various embodiments.

FIG. 12 illustrates non-limiting reversal of established incision-induced hyperalgesia by Compound 1 and the finding that a daily dose of Compound 1 prevents the return to hyperalgesia, in accordance with various embodiments.

FIG. 13 illustrates non-limiting prevention of hyperalgesia following a severe incisional injury by daily dosing of Compound 1, in accordance with various embodiments.

FIG. 14 illustrates non-limiting reversal of mechanical hypersensitivity by Compound 1 in a diabetic neuropathy model, in accordance with various embodiments. Streptozotocin (STZ) destroys insulin-producing cells and generates a diabetic phenotype in mice. Animals were dosed with STZ on day −7. By day 0 animals are hyperglycemic and hyperalgesic (day 0 BL). Compound 1 blocks STZ-induced mechanical allodynia. Upon repeated dosing, Compound 1 is similar in efficacy and potency to gabapentin despite being peripherally restricted.

FIG. 15 illustrates Compound 1 concentration in dog plasma from a single dose PO study, in accordance with various embodiments.

FIG. 16 illustrates Compound 1 concentration in dog plasma from a single dose IV study, in accordance with various embodiments.

FIG. 17 illustrates an XPRD spectrum of amorphous Compound 1.

FIG. 18 illustrates a comparison of the XPRD spectra of Compound 1 free base (top trace) and Compound 1 (bottom trace).

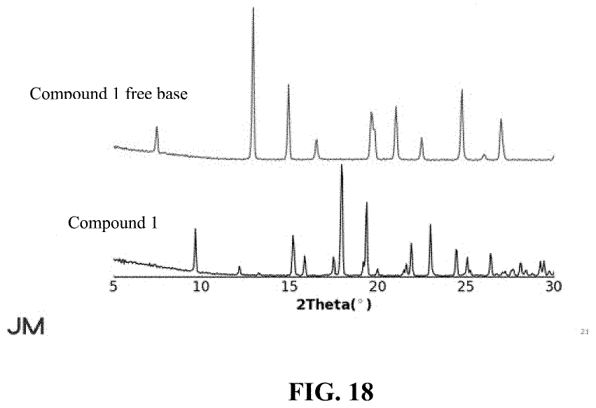

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X², and X³ are all different, where X¹ and X² are the same but X³ is different, and any other analogous permutations.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds,” as used herein, is meant that a first molecule preferentially binds to a second molecule (e.g., a particular receptor or enzyme), but does not necessarily bind only to that second molecule.

As used herein, a “subject,” “individual,” or “patient” may be a human or non-human mammal or a bird. Non-human mammals include, for example, livestock and pets, such as ovine, bovine, porcine, canine, feline and murine mammals. Preferably, the individual is human. The term “individual” as used herein, also refers to an individual or a subject, a patient or a person in need of relief of pain, or a human volunteer willing to be administered a therapeutic agent.

The term “treat,” “treating,” or “treatment,” as used herein, means reducing the frequency or severity with which symptoms of a disease or condition are experienced by a subject by virtue of administering an agent or compound to the subject.

The term “incision” as used herein means any puncturing of the individual's skin, by surgical cutting instruments, laparoscopic instruments, stitches, pacemakers, and the like. The term “incision” also encompasses internal (in the body cavity of the individual) cuts, cauterizations, stitches, or other medical procedures performed during surgery. The term “incision” also encompasses external punctures to the skin caused outside a medical setting that were subsequently treated medically or surgically.

The following abbreviations are used herein: BBr₃, boron tribromide; CD₃OD, (tetra)deuterio-methanol; COX, cyclooxygenase; d, day(s); DMSO, dimethylsulfoxide; DSC, differential scanning calorimetry; ELSD, evaporative light-scattering detection; g, gram; GC, gas chromatography; GC-MS, gas chromatography-mass spectrometry; GVS, gravimetric vapor sorption; h, hour(s); HCl, hydrochloric acid; HPLC, high performance liquid chromatography; ICH, International Conference on Harmonisation; iPrOH, isopropanol; IR, infrared (spectrum); mg, milligram; min, minute(s); mL, milliliter; mol, mole; mmol, millimole; MTBE, methyl tert-butyl ether; NADPH, dihydronicotinamide-adenine dinucleotide phosphate; NaOH, sodium hydroxide; ng, nanogram; NLT, not less than; NMR, nuclear magnetic resonance; NMT, not more than; NOS, nitric oxide synthase; NSAID, non-steroidal anti-inflammatory drug; pKa, negative base-10 logarithm of the acid dissociated constant; PN, peroxynitrite; RNS, reactive nitrogen species; ROI, residue on ignition; ROS, reactive oxygen species; TRP, Transient-Receptor Potential; USP, United States Pharmacopeia; UV, ultraviolet;)(RFD, x-ray (powder) diffraction pattern.

### Method of Treating Diabetic Neuropathy

In various embodiments, a method of treating diabetic neuropathy is provided. The method includes administering a therapeutically effective amount of a composition that includes a compound of Formula I:

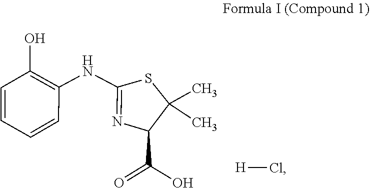 [SMILES: CC1(C)SC(Nc2ccccc2O)=N[C@@H]1C(=O)O ; Cl]

to an individual having diabetic neuropathy. The formal name of Compound 1 is (R)-2-(2-hydroxyphenylamino)-5,5-dimethyl-4,5-dihydrothiazole-4-carboxylic acid mono-hydrochloride. Although Compound 1 is crystalline, the amorphous form of Compound 1 can also be used in the method of treating diabetic neuropathy described herein. Additionally, a mixture of crystalline Compound 1 and amorphous Compound 1, in any proportions, can also be used in the method of treating diabetic neuropathy described herein. The enantiomer of Compound 1 is (S)-2-(2-hydroxyphenylamino)-5,5-dimethyl-4,5-dihydrothiazole-4-carboxylic acid mono-hydrochloride, and can be designated (S)-Compound 1. In various embodiments, the enantiomeric purity of Compound 1 can be at least about 95%, 97%, 98%, 99%, 99.2%, 99.4%, 99.6%, 98.8%, 99.9%, 99.99%, or more. Thus, for example, if the enantiomeric purity of Compound 1 is 99.5%, the composition contains 99.5% Compound 1 and 0.5% (5)-Compound 1. The enantiomeric purity refers only to the relative amounts of Compound 1 and (5)-Compound 1, and additional impurities may be present as described herein. In various embodiments, the composition includes a therapeutically effective amount of a racemic mixture of Compound 1. A racemic mixture of Compound 1 contains about 50% Compound 1 and about 50% (5)-Compound 1. The method can be used to treat diabetic neuropathy in individuals having either type I or type II diabetes.

The method can be used to treat peripheral neuropathy, which is the most common form of neuropathy linked to diabetes. Peripheral neuropathy is often associated with damage to the nerves leading to an individual's feet, and can result in foot deformities, infections, ulcers, and amputations. The method can also be used to treat proximal neuropathy, which is also called diabetic amyotrophy. This form of neuropathy specifically affects the muscles in the upper part of the leg(s), buttocks, and hips. Proximal neuropathy can also involve nerve pain, especially pain that shoots from the lower back and down the leg, which is called radiculopathy (sciatica). Proximal neuropathy often affects elderly people with diabetes.

The method can also be used to treat autonomic neuropathy in a diabetic individual, which affects the autonomic nerves responsible for maintaining unconscious bodily functions such as pumping of the heart, breathing, and digestion. Autonomic neuropathy can be particularly severe because it can affect many of the body's systems, from the digestive tract to vision.

The method can also be used to treat focal neuropathy in a diabetic individual. Focal neuropathy affects a specific nerve rather than many nerves. Focal neuropathy, which often comes on suddenly, most often affects nerves in the head (especially nerves that connect to the eyes). It can also affect the torso and legs. When focal neuropathy affects the legs, it has different symptoms than proximal neuropathy. Proximal neuropathy causes muscle weakness in the legs, and it can also cause shooting pain down the leg. Focal neuropathy, however, causes pain in very specific locations on the legs.

The method can also be used to treat pain occurring during progression of neurodegenerative diseases such as amyotrophic lateral sclerosis (ALS), Parkinson's Disease, Multiple Sclerosis, as well as neurotraumatic events such as stroke and ischemia.

Dosing and Dosing Regimens for Treatment of Diabetic Neuropathy

In various embodiments, the therapeutically effective amount of Compound 1 for treating diabetic neuropathy can be from about 5 mg to about 5000 mg. The therapeutically effective amount of Compound 1 can be about 10 mg to about 4750 mg, about 25 mg to about 4500 mg, about 50 mg to about 4250 mg, about 100 mg to about 4000 mg, about 150 mg to about 3750 mg, about 200 mg to about 3500 mg, about 275 mg to about 3250 mg, or about 100 mg to about 3000 mg, about 200 mg to about 2000 mg, or about 300 mg to 1000 mg. In various embodiments, the therapeutically effective amount of Compound 1 can be at least, equal to, or greater than about 5 mg, 10 mg, 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 600 mg, 750 mg, 1000 mg, 1250 mg, 1500 mg, 1750 mg, 2000 mg, 2500 mg and 3000 mg.

The therapeutically effective amount of Compound 1 can be administered once a day, twice a day, three times a day, four times a day, or more. In various embodiments, the therapeutically effective amount of Compound 1 is administered for about 1 day to about 90 days. The therapeutically effective amount of Compound 1 can be administered for about 1 day, 2 days, 3 days, 4 days, 5 days, 6 days, 7 days, 14 days, 28 days, or more. Administering of Compound 1 can continue for as long as the individual, in consultation with a physician, deems it necessary to maintain adequate pain control for their individual situation. In various embodiments, Compound 1 can be administered for about 1 month to about 24 months, or for the lifespan of the individual.

### Method of Treating Post-Surgical Pain

In various embodiments, a method of treating post-surgical pain is provided. The method includes administering a therapeutically effective amount of a composition that includes a compound of Formula I:

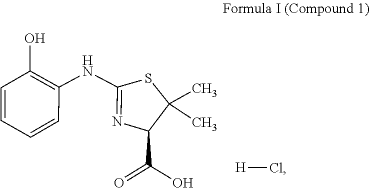 [SMILES: CC1(C)SC(Nc2ccccc2O)=N[C@@H]1C(=O)O ; Cl]

to an individual having post-surgical pain. Although Compound 1 is crystalline, the amorphous form of Compound 1 can also be used in the method of treating post-surgical pain described herein. Additionally, a mixture of crystalline Compound 1 and amorphous Compound 1, in any proportions, can also be used in the method of treating post-surgical pain described herein. The enantiomer of Compound 1 is (S)-2-(2-hydroxyphenylamino)-5,5-dimethyl-4,5-dihydrothiazole-4-carboxylic acid mono-hydrochloride, and can be designated (9-Compound 1. In various embodiments, the enantiomeric purity of Compound 1 can be at least about 95%, 97%, 98%, 99%, 99.2%, 99.4%, 99.6%, 98.8%, 99.9%, 99.99%, or more. Thus, for example, if the enantiomeric purity of Compound 1 is 99.5%, the composition contains 99.5% Compound 1 and 0.5% (9-Compound 1. The enantiomeric purity refers only to the relative amounts of Compound 1 and (9-Compound 1, and additional impurities may be present as described herein. In various embodiments, the composition includes a therapeutically effective amount of a racemic mixture of Compound 1. A racemic mixture of Compound 1 contains about 50% Compound 1 and about 50% (9-Compound 1. The method can be used to treat pain resulting from surgery. Generally, individuals become aware of post-surgical pain after any general or local anesthetic the individual received prior to or during a surgical procedure wears off. In various embodiments, the post-surgical pain is present at or near at least one surgical site. The surgical site can be one or more locations on the surface of the individual and/or within the body cavity of the individual. In various embodiments, the surgical site includes at least one incision.

Compound 1 can be used, without limitation, in acute and sub-acute setting (duration<14 days) as a non-opioid treatment of pain including in peri-operative settings as a replacement for ‘gateway’ opioids products (e.g., PERCOCET®, VICODIN®) often prescribed following surgical procedures. Compound 1 can be used to treat post-surgical pain from any type of surgery or procedure, non-limiting examples of which include appendectomy, arthroscopic surgery, brain surgery, breast biopsy, carotid endarterectomy, cataract surgery, Cesarean section, cholecystectomy, circumcision, coronary artery bypass, colon or rectal, debridement of wound, burn, or infection, dilation and curettage, endoscopy, free skin graft, gastric bypass, hemorrhoidectomy, hip replacement, hysterectomy, hysteroscopy, inguinal hernia repair, knee replacement, laparoscopic procedures, low back pain surgery, liver resection, lung resection, mastectomy (partial, total, or modified radical), mediport insertion or removal, orthopedic surgery, partial colectomy, parathyroidectomy, prostatectomy, spinal surgery, third-molar extraction, tooth extraction, tubal ligation, thyroidectomy, and tonsillectomy.

### Dosing and Dosing Regimens for Treatment of Post-Surgical Pain

In various embodiments, the therapeutically effective amount of Compound 1 for treating post-surgical pain can be from about 5 mg to about 5000 mg. The therapeutically effective amount of Compound 1 can be about 10 mg to about 4750 mg, about 25 mg to about 4500 mg, about 50 mg to about 4250 mg, about 100 mg to about 4000 mg, about 150 mg to about 3750 mg, about 200 mg to about 3500 mg, about 275 mg to about 3250 mg, or about 100 mg to about 3000 mg, about 200 mg to about 2000 mg, or about 300 mg to 1000 mg. In various embodiments, the therapeutically effective amount of Compound 1 can be at least, equal to, or greater than about 5 mg, 10 mg, 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 600 mg, 750 mg, 1000 mg, 1250 mg, 1500 mg, 1750 mg, 2000 mg, 2500 mg and 3000 mg.

The therapeutically effective amount of Compound 1 can be administered once a day, twice a day, three times a day, four times a day, or more. In various embodiments, the therapeutically effective amount of Compound 1 is administered for about 1 day to about 90 days. The therapeutically effective amount of Compound 1 can be administered for about 1 day, 2 days, 3 days, 4 days, 5 days, 6 days, 7 days, 14 days, 28 days, or more. Administering of Compound 1 can continue for as long as the individual, in consultation with a physician, deems it necessary to maintain adequate pain control for their individual situation. In various embodiments, Compound 1 can be administered for about 1 month to about 24 months, or for the lifespan of the individual.

In various embodiments, administering Compound 1 under any of the conditions described herein can result in a maximum observed plasma concentration (Cₘₐₓ) of about 5 μg/mL to about 300 μg/mL in a rat, mouse, dog, or human. The Cₘₐₓ of Compound 1 can be about 10 μg/mL to about 280 μg/mL, about 20 μg/mL to about 260 μg/mL, about 40 μg/mL to about 240 μg/mL, about 50 μg/mL to about 220 μg/mL, about 60 μg/mL to about 200 μg/mL, about 70 μg/mL to about 180 μg/mL, about 80 μg/mL to about 160 μg/mL, about 90 μg/mL to about 140 μg/mL, or about 95 μg/mL to about 120 μg/mL. In various embodiments, the Cₘₐₓ of Compound 1 can be at least, equal to, or greater than about 5 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 220 μg/mL, 240 μg/mL, 260 μg/mL, 280 μg/mL, or about 300 μg/mL.

In various embodiments, administering Compound 1 under any of the conditions described herein can result in an area under the curve (AUC_{INF}) of about 100 hr·μg/mL to about 3000 hr·μg/mL in a rat, mouse, dog, or human. The AUC_{INF} of Compound 1 can be about 100 hr·μg/mL to about 2800 hr·μg/mL, about 200 hr·μg/mL to about 2600 hr·μg/mL, about 400 hr·μg/mL to about 2400 hr·μg/mL, about 500 hr·μg/mL to about 2200 hr·μg/mL, about 600 hr·μg/mL to about 2000 hr·μg/mL, about 700 hr·μg/mL to about 1800 hr·μg/mL, about 800 hr·μg/mL to about 1600 hr·μg/mL, about 900 hr·μg/mL to about 1400 hr·μg/mL, or about 950 hr·μg/mL to about 1200 hr·μg/mL. In various embodiments, the AUC_{INF} of Compound 1 can be at least, equal to, or greater than about 50 hr·μg/mL, 100 hr·μg/mL, 200 hr·μg/mL, 300 hr·μg/mL, 400 hr·μg/mL, 500 hr·μg/mL, 600 hr·μg/mL, 700 hr·μg/mL, 800 hr·μg/mL, 900 hr·μg/mL, 1000 hr·μg/mL, 1200 hr·μg/mL, 1400 hr·μg/mL, 1600 hr·μg/mL, 1800 hr·μg/mL, 2000 hr·μg/mL, 2200 hr·μg/mL, 2400 hr·μg/mL, 2600 hr·μg/mL, 2800 hr·μg/mL, or about 3000 hr·μg/mL.

Combination Therapies

In various embodiments, the method includes administering a therapeutically effective amount of a composition containing Compound 1 in combination or adjunctively with at least one additional pharmaceutically active agent. The type of pharmaceutically active agent that can be administered in combination or adjunctively with Compound 1 is not particularly limited. Non-limiting examples of additional pharmaceutically active agents include acetaminophen, alpha-2 adrenergic agonists, aspirin, COX-1 inhibitors, COX-2 inhibitors, voltage-gated ion channel blockers (NaV, CaV and KaV families), ligand-gated ion channels (TRPV1, TRPV4, TRPA1, and TRPM8 antagonists and agonists), opioid analgesics (mu-, delta-, kappa-selective and mixed), non-opioid analgesics, non-steroidal anti-inflammatories, norepinephrine reuptake inhibitors, serotonin reuptake inhibitors, dual norepinephrine-serotonin reuptake inhibitors, anticonvulsants (lamotrigine) including the gabapentinoids (gabapentin, pregabalin, mirogabalin), antidepressants (including tricyclics such as amitriptyline, doxepin and desipramine), tramadol and tapentadol.

Non-limiting examples of analgesic drugs that can be useful in combination or adjunctive therapy with Compound 1 include without limitation acetaminophen, alfentanil, allylprodine, alphaprodine, anileridine, aspirin, benzylmorphine, bezitramide, buprenorphine, butorphanol, clonidine, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, duloxetine, eptazocine, ethoheptazine, ethylmethylthiambutene, ethylmorphine, etonitazene, fentanyl, gabapentin, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, mirogabalin, morphine, myrophine, nalbuphine, nalorphine, narceine, nicomorphine, norlevorphanol, normethadone, normorphine, norpipanone, opium, oxycodone, oxymorphone, papaveretum, pentazocine, phenadoxone, phenazocine, phenomorphan, phenoperidine, piminodine, piritramide, pregabalin, proheptazine, promedol, properidine, propiram, propoxyphene, sufentanil, tapentadol, tilidine, tramadol, NO-naproxen, NCX-701, ALGRX-4975, pharmaceutically acceptable salts thereof, and any combinations thereof.

Non-limiting examples of anticonvulsants that can be useful in combination or adjunctively with Compound 1 include without limitation acetylpheneturide, albutoin, aminoglutethimide, 4-amino-3-hydroxybutyric acid, atrolactamide, beclamide, buramate, carbamazepine, cinromide, clomethiazole, clonazepam, decimemide, diethadione, dimethadione, doxenitoin, eterobarb, ethadione, ethosuximide, ethotoin, felbamate, fluoresone, fosphenyloin, gabapentin, ganaxolone, lamotrigine, levetiracetam, lorazepam, mephenyloin, mephobarbital, metharbital, methetoin, methsuximide, midazolam, mirogabalin, narcobarbital, nitrazepam, oxcarbazepine, paramethadione, phenacemide, phenetharbital, pheneturide, phenobarbital, phensuximide, phenylmethylbarbituric acid, phenyloin, phenethylate, pregabalin, primidone, progabide, remacemide, rufinamide, suclofenide, sulthiame, talampanel, tetrantoin, tiagabine, topiramate, trimethadione, valproic acid, valpromide, vigabatrin, zonisamide, pharmaceutically acceptable salts thereof, and any combinations thereof.

Non-limiting examples of antidepressants that can be useful in combination or adjunctively with Compound 1 include without limitation bicyclic, tricyclic and tetracyclic antidepressants, hydrazides, hydrazines, phenyloxazolidinones and pyrrolidones. Specific examples include adinazolam, adrafinil, amineptine, amitriptyline, amitriptylinoxide, amoxapine, befloxatone, bupropion, butacetin, butriptyline, caroxazone, citalopram, clomipramine, cotinine, demexiptiline, desipramine, dibenzepin, dimetacrine, dimethazan, dioxadrol, dothiepin, doxepin, duloxetine, etoperidone, femoxetine, fencamine, fenpentadiol, fluacizine, fluoxetine, fluvoxamine, hematoporphyrin, hypericin, imipramine, imipramine N-oxide, indalpine, indeloxazine, iprindole, iproclozide, iproniazid, isocarboxazid, levophacetoperane, lofepramine, maprotiline, medifoxamine, melitracen, metapramine, metralindole, mianserin, milnacipran, minaprine, mirtazapine, moclobemide, nefazodone, nefopam, nialamide, nomifensine, nortriptyline, noxiptilin, octamoxin, opipramol, oxaflozane, oxitriptan, oxypertine, paroxetine, phenelzine, piberaline, pizotyline, prolintane, propizepine, protriptyline, pyrisuccideanol, quinupramine, reboxetine, ritanserin, roxindole, rubidium chloride, sertraline, sulpiride, tandospirone, thiazesim, thozalinone, tianeptine, tofenacin, toloxatone, tranylcypromine, trazodone, trimipramine, tryptophan, venlafaxine, viloxazine, zimeldine, pharmaceutically acceptable salts thereof, and any combinations thereof.

The additional pharmaceutically active agent can be included with Compound 1 in the same dosage form or in a separate dosage form, and any of the dosage forms described herein can be suitably used for combining Compound 1 and an additional pharmaceutically active agent in the same dosage form. When the additional pharmaceutically active agent is present in a separate dosage form, the additional pharmaceutically active agent can be administered at the same time as Compound 1 or at a different time, such as about 1 hour to about 24 hours after administration of Compound 1. The additional pharmaceutically active agent can be administered for the entire duration of administration of Compound 1, or for a shorter or longer time.

### Dosage Forms and Formulations

In various embodiments, the composition can include at least one pharmaceutically acceptable carrier and/or at least one pharmaceutically acceptable excipient.

Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 18^{th} Edition (1990, Mack Publication Co., New Jersey).

The composition can be prepared, packaged, or sold in the form of a sterile injectable aqueous or oily suspension or solution. This suspension or solution may be formulated according to the known art, and may comprise, in addition to the active ingredient, additional ingredients such as anti-oxidants, dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Compositions that are useful in the methods described herein can be administered, prepared, packaged, and/or sold in formulations suitable for intravenous, subcutaneous, sublingual, oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions can be administered via numerous routes, including, but not limited to, intravenous, subcutaneous, sublingual, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disorder being treated, the type and age of the veterinary or human patient being treated, and the like.

Compositions that are useful in the methods described herein can be administered systemically in intravenous and subcutaneous liquid formulations, oral and sublingual solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer compounds according to the methods of the invention.

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

The formulations of the compositions described herein can be prepared by any method known or hereafter developed in the art of pharmacology. In general, such preparatory methods include the step of bringing the active ingredient (e.g., Compound 1) into association with a carrier or one or more other accessory ingredients, and then, if necessary or desirable, shaping or packaging the product into a desired single- or multi-dose unit.

Although the descriptions of compositions provided herein are principally directed to pharmaceutical compositions which are suitable for ethical administration to humans, it will be understood by the skilled artisan that such compositions are generally suitable for administration to subjects of all sorts.

Modification of compositions suitable for administration to humans in order to render the compositions suitable for administration to various animals is well understood, and the ordinarily skilled veterinary pharmacologist can design and perform such modification with merely ordinary, if any, experimentation. Subjects to which administration of the compositions described herein are contemplated include, but are not limited to, humans and other primates, mammals including commercially relevant mammals such as cattle, pigs, horses, sheep, cats, and dogs.

Compositions that are useful in the methods described herein can be prepared, packaged, or sold in formulations suitable for intravenous, subcutaneous, sublingual, oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A composition for use in the methods described herein can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient (e.g., Compound 1), the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Controlled- or sustained-release formulations of a pharmaceutical composition of the invention may be made using conventional technology. Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation can be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, polar lipids, or N-methyl-2-pyrrolidone.

One acceptable vehicle for topical delivery of some of the compositions of the invention may contain liposomes. The composition of the liposomes and their use are known in the art (for example, see U.S. Pat. No. 6,323,219).

A topical dosage form of Compound 1 can be optionally combined with other ingredients such as adjuvants, anti-oxidants, chelating agents, surfactants, foaming agents, wetting agents, emulsifying agents, viscosifiers, buffering agents, preservatives, and the like. In various embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

A topical dosage form of Compound 1 should be applied in an amount effective to affect desired changes. As used herein “amount effective” shall mean an amount sufficient to cover the region of skin surface where a change is desired. In various embodiments, Compound 1 can be present in the amount of from about 0.0001% to about 15% by weight volume of the composition. In various embodiments, Compound 1 can be present in an amount from about 0.0005% to about 5% of the composition; most preferably, it should be present in an amount of from about 0.001% to about 1% of the composition.

Liquid derivatives and natural extracts made directly from biological sources may be employed in the compositions described herein in a concentration (w/v) from about 1 to about 99%. Fractions of natural extracts and protease inhibitors may have a different preferred range, from about 0.01% to about 20% and, more preferably, from about 1% to about 10% of the composition. Of course, mixtures of the active agents described herein can be combined and used together in the same formulation, or in serial applications of different formulations.

The compositions described herein can include a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition described herein such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition can include an antioxidant and a chelating agent which can inhibit any the degradation of Compound 1 that may occur, for use in the invention in the aqueous gel formulation. Suitable antioxidants include BHT, BHA, α-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art. Controlled-release preparations may also be used and the methods for the use of such preparations are known to those of skill in the art.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Suitable dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Suitable emulsifying agents include, but are not limited to, lecithin, and acacia. Suitable preservatives include, but are not limited to, methyl, ethyl, or n-propyl-para-hydroxybenzoates, ascorbic acid, and sorbic acid. Suitable sweetening agents include, for example, glycerol, propylene glycol, sorbitol, sucrose, and saccharin. Suitable thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

The composition described herein can also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, an “oily” liquid is one which comprises a carbon-containing liquid molecule and which exhibits a less polar character than water.

A formulation of the compositions described herein suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The terms oral rinse and mouthwash are used interchangeably herein.

Compositions as described herein can be prepared, packaged, or sold in a formulation suitable for oral or buccal administration. Such a formulation can include, but is not limited to, a gel, a liquid, a suspension, a paste, toothpaste, a mouthwash or oral rinse, and a coating. For example, an oral rinse of the invention may comprise a compound of the invention at about 1.4%, chlorhexidine gluconate (0.12%), ethanol (11.2%), sodium saccharin (0.15%), FD&C Blue No. 1 (0.001%), peppermint oil (0.5%), glycerine (10.0%), Tween 60 (0.3%), and water to 100%. In another embodiment, a toothpaste of the invention may comprise a compound of the invention at about 5.5%, sorbitol, 70% in water (25.0%), sodium saccharin (0.15%), sodium lauryl sulfate (1.75%), carbopol 934, 6% dispersion in (15%), oil of spearmint (1.0%), sodium hydroxide, 50% in water (0.76%), dibasic calcium phosphate dihydrate (45%), and water to 100%. The examples of formulations described herein are not exhaustive, and it is understood that the invention includes additional modifications of these and other formulations not described herein, but which are known to those of skill in the art.

A tablet that includes Compound 1 can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Suitable dispersing agents include, but are not limited to, potato starch and sodium starch glycollate. Suitable surface-active agents include, but are not limited to, sodium lauryl sulphate. Suitable diluents include, but are not limited to, calcium carbonate, sodium carbonate, lactose, microcrystalline cellulose, calcium phosphate, calcium hydrogen phosphate, and sodium phosphate. Suitable granulating and disintegrating agents include, but are not limited to, corn starch and alginic acid. Suitable binding agents include, but are not limited to, gelatin, acacia, pre-gelatinized maize starch, polyvinylpyrrolidone, and hydroxypropyl methylcellulose. Suitable lubricating agents include, but are not limited to, magnesium stearate, stearic acid, silica, and talc.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules that include Compound 1 can be made using a physiologically degradable composition, such as gelatin. Such hard capsules include Compound 1, and can further include additional ingredients including, for example, an inert solid diluent such as calcium carbonate, calcium phosphate, or kaolin.

Soft gelatin capsules that include Compound 1 can be made using a physiologically degradable composition, such as gelatin. Such soft capsules include Compound 1, which may be mixed with water or an oil medium such as peanut oil, liquid paraffin, or olive oil.

Liquid formulations of compositions described herein which are suitable for oral administration can be prepared, packaged, and sold either in liquid form or in the form of a dry product intended for reconstitution with water or another suitable vehicle prior to use.

Compositions described herein can be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining Compound 1 with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

Formulations of a pharmaceutical composition suitable for parenteral administration include the active ingredient (e.g. Compound 1) combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as antioxidants, dispersing agents, wetting agents, or suspending agents described herein. Such sterile injectable formulations can be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Compositions described herein can be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may include a powder or an aerosolized or atomized solution or suspension including the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compositions described herein can be administered to a subject, preferably a human, will vary depending upon any number of factors, including but not limited to, the type of animal and type of disease state being treated, the age of the subject and the route of administration.

### Kits

In various embodiments, a kit is provided. The kit includes a composition comprising a compound of Formula I,

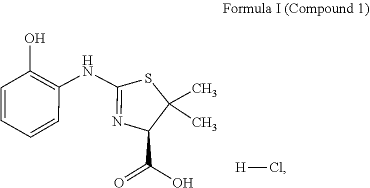 [SMILES: CC1(C)SC(Nc2ccccc2O)=N[C@@H]1C(=O)O ; Cl]

an applicator, and instructional material for use thereof.

The instructional material comprises instructions for treating diabetic neuropathy or post-surgical pain. In various embodiments, the instructional material includes instructions for administering the composition comprising about 5 mg to about 500 mg of Compound 1. In various embodiments, the instructional material includes instructions for treating diabetic neuropathy. In various embodiments, the instructional material comprises instructions for treating post-surgical pain.

### EXAMPLES

Various embodiments of the present invention can be better understood by reference to the following Examples, which are offered by way of illustration. The present invention is not limited to the Examples given herein.

### Example 1: Physical Properties of Compound 1

Compound 1, (R)-2-(2-hydroxyphenylamino)-5,5-dimethyl-4,5-dihydrothiazole-4-carboxylic acid mono-hydrochloride, has the structure of Formula I:

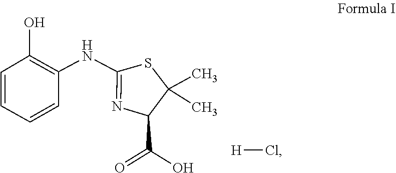 [SMILES: CC1(C)SC(Nc2ccccc2O)=N[C@@H]1C(=O)O ; Cl]

Compound 1 has the following pKa values: 2.29±0.02 (Acidic), 6.97±0.01 (Basic), and 10.24±0.03 (Acidic). Compound 1 is freely soluble in methanol and tert-butyl alcohol:water (1:1). Compound 1 is sparingly soluble in 2-propanol, ethanol, 10% water:isopropyl acetate, 10% water/tetrahydrofuran, and water. Compound 1 is less than sparingly soluble in n-heptane, toluene, acetone, tetrahydrofuran, ethyl acetate, isopropyl acetate, t-butyl methyl ether, and t-butyl alcohol.

Compound 1 has a LogD distribution coefficient at pH 7.2 of −0.07 (3 mL PBS Buffer: 1 mL Octanol) and −0.39 (2 mL PBS Buffer: 2 mL Octanol), where PBS is phosphate buffer solution.

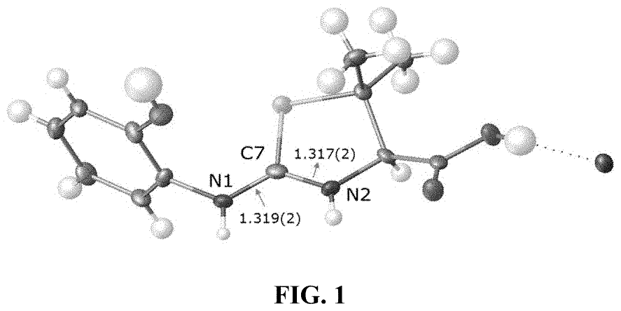

FIG. 1 shows the X-ray crystal structure of Compound 1. The crystallographic parameters for the structure in FIG. 1 are listed in Table 1 below.

TABLE 1
Crystal Data for (R)-2-(2-hydroxyphenylamino)-5,5-dimethyl-
4,5-dihydrothiazole-4-carboxylic acid mono-hydrochloride

| Crystal System | Orthorhombic | |
|---|---|---|
| Space Group | P212121 | |
| Unit Cell Dimensions | a = 7.00762(9) Å | α = 90° |
| | b = 10.08020(10) Å | β = 90° |
| | c = 20.5203(2) Å | γ = 90° |
| | Volume = 1449.52(3) Å³ | |
| Goodness of Fit on F² | 1.046 | |
| Z′ | 4 | |

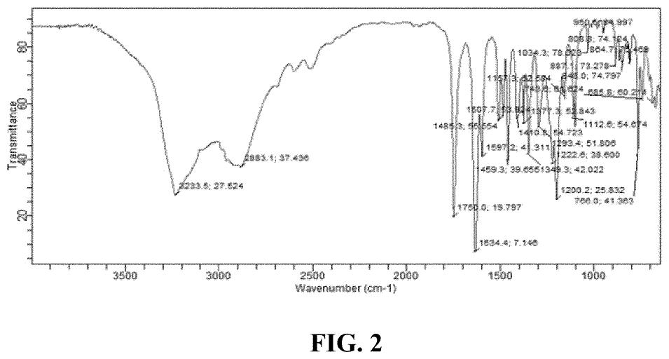

FIG. 2 is the infrared spectrum of Compound 1. Table 2 lists the peak assignments of the functional groups in Compound observed in the infrared spectrum of Compound 1.

TABLE 2
Interpretation of (R)-2-(2-hydroxyphenylamino)-5,5-dimethyl-4,5-
dihydrothiazole-4-carboxylic acid mono-hydrochloride IR Data

| Range of | | | |
|---|---|---|---|
| Absorption | | | Type of |
| (cm⁻¹) | Functional Group | Intensity | Vibrations |
| *3200-3300 | N—H (Amine) | Broad | N—H Stretching |
| 2830-3000 | O—H (Acid) | Very broad | O—H Stretching |
| 1690-1750 | C═O (Carbonyl) | Sharp | C═O Stretching |
| 1590-1650 | C═N | Sharp | C═N Stretching |
| 1400-1600 | C═C | Medium | C═C Stretching |
| | | | (Aromatic) |

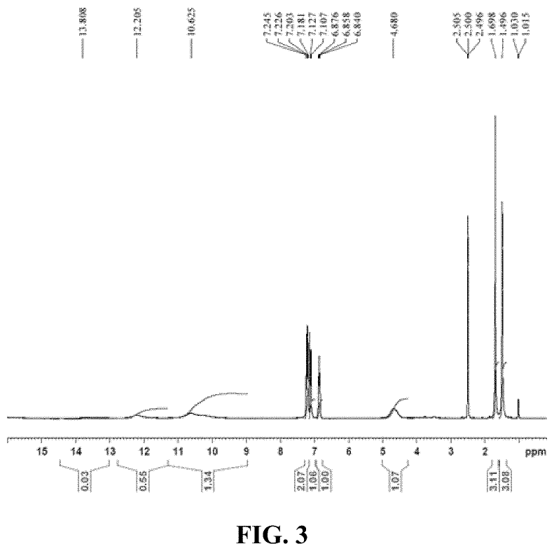

FIG. 3 shows the ¹H NMR spectrum of Compound 1. Table 3 lists the peak assignments for the hydrogen nuclei in Compound 1.

TABLE 3
Interpretation of ¹H-NMR Spectrum of
(R)-2-(2-hydroxyphenylamino)-5,5-dimethyl-4,5-
dihydrothiazole-4-carboxylic acid mono-hydrochloride

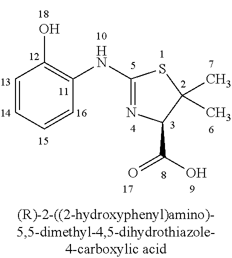 [SMILES: CC1(C)SC(Nc2ccccc2O)=N[C@@H]1C(=O)O]

| Chemical Shift | | Proton | Total Proton |
|---|---|---|---|
| (ppm) | Multiplicity | Number | Integration |
| 12.205 | Broad singlet | OH | 1 |
| 10.625 | Broad singlet | NH | 1 |
| 7.245-7.181 | multiplet | 14&16 | 2 |
| 7.127-7.107(J = 8) | doublet | 13 | 1 |
| 6.876-6.840 (J = 7.2) | triplet | 15 | 1 |
| 4.680 | singlet | 3 | 1 |
| 1.698 | Singlet | 6 | 3 |
| 1.496 | Singlet | 7 | 3 |

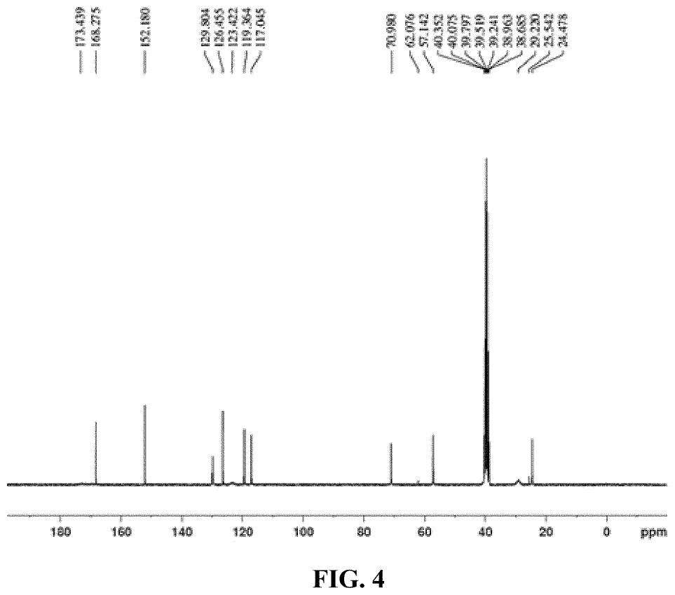

FIG. 4 shows the ¹³C NMR spectrum of Compound 1. Table 4 lists the peak assignments for the carbon nuclei in Compound 1.

TABLE 4
Interpretation of ¹³C-NMR Spectrum of (R)-2-
(2-hydroxyphenylamino)-5,5-dimethyl-4,5-dihydrothiazole-
4-carboxylic acid mono-hydrochloride

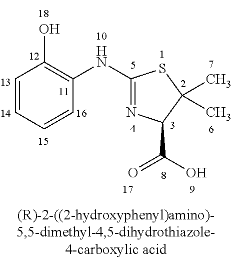 [SMILES: CC1(C)SC(Nc2ccccc2O)=N[C@@H]1C(=O)O]

| Chemical Shift (ppm) | Assignment | Number of Carbons | Type of Carbon |
|---|---|---|---|
| 24.48 | 6 | 1 | Primary |
| 29.22 | 7 | 1 | Primary |
| 57.14 | 2 | 1 | Quaternary |
| 70.98 | 3 | 1 | Tertiary |
| 117.05 | 13 | 1 | Tertiary |
| 119.36 | 15 | 1 | Tertiary |
| 123.42 | 11 | 1 | Quaternary |
| 126.46 | 16 | 1 | Tertiary |
| 129.80 | 14 | 1 | Tertiary |
| 152.18 | 12 | 1 | Quaternary |
| 168.28 | 8 | 1 | Quaternary |
| 173.44 | 5 | 1 | Quaternary |

Additional characteristics of Compound 1 and related compounds are described in U.S. Pat. No. 9,102,636, which is hereby incorporated by reference in its entirety.

### Example 2: Polymorphs of Compound 1

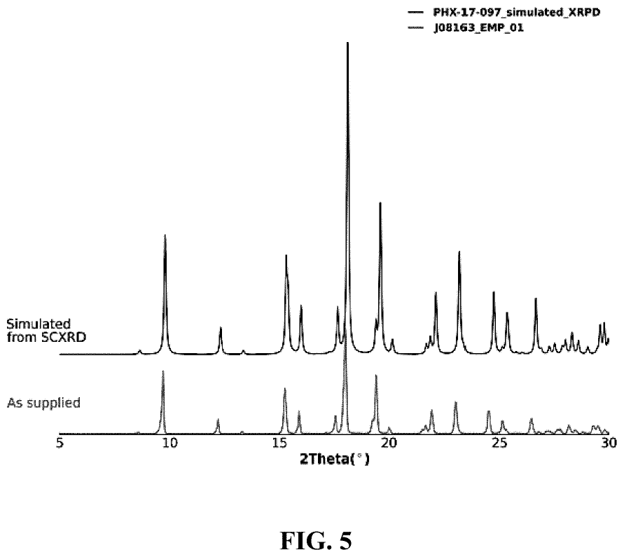

Polymorphic screening was performed using 15 organic/aqueous solvent systems, including: n-heptane, methanol, toluene, acetone, tetrahydrofuran, 2-propanol, ethanol, ethyl acetate, iso-propyl acetate, tert-butylmethyl ether, 10% water/90% iso-propyl alcohol, 10% water/90% tetrahydrofuran, tert-butyl alcohol, water, and 1:1 tert-butyl alcohol:water. Only one crystalline form was obtained (Form 1). Compound 1 is a non-solvated, crystalline, mono HCl salt. FIG. 5 shows the experimentally obtained XPRD spectrum of Compound 1 in the bottom trace, and the simulated XPRD spectrum in the top trace. The experimentally obtained XPRD spectrum of Compound 1 has the following peaks and associated intensities:

| Angle (2-Theta) | Intensity % |
|---|---|
| 9.6 | 43.3 |
| 12.2 | 10.7 |
| 13.3 | 4.5 |
| 15.2 | 37.6 |
| 15.8 | 19.9 |
| 17.5 | 18.7 |
| 18.0 | 100.0 |
| 19.2 | 14.8 |
| 19.4 | 66.6 |
| 20.0 | 8.3 |
| 21.5 | 7.2 |
| 21.7 | 12.6 |
| 21.9 | 31.0 |
| 23.0 | 47.6 |
| 24.5 | 25.2 |
| 25.1 | 18.6 |
| 25.2 | 6.9 |
| 26.4 | 21.2 |
| 26.7 | 4.1 |
| 27.1 | 5.4 |
| 27.2 | 6.4 |
| 27.7 | 8.1 |
| 28.1 | 13.2 |
| 28.4 | 6.7 |
| 28.8 | 4.1 |
| 29.2 | 15.1 |
| 29.4 | 15.1 |
| 29.7 | 6.0 |
| 30.1 | 12.3 |
| 30.5 | 12.2 |
| 31.1 | 13.8 |
| 31.4 | 26.6 |
| 31.9 | 11.4 |
| 32.8 | 7.6 |
| 34.0 | 15.5 |
| 34.5 | 7.5 |
| 35.1 | 4.8 |
| 35.4 | 6.6 |
| 35.7 | 5.0 |
| 36.4 | 6.9 |
| 36.9 | 3.8 |
| 37.5 | 13.8 |
| 37.7 | 8.3 |
| 38.0 | 4.8 |
| 38.5 | 6.6 |
| 39.0 | 5.6 |
| 39.3 | 15.5 |
| 39.7 | 3.1 |
| 40.3 | 5.1 |
| 40.6 | 5.4 |
| 40.7 | 5.3 |
| 41.5 | 6.7 |

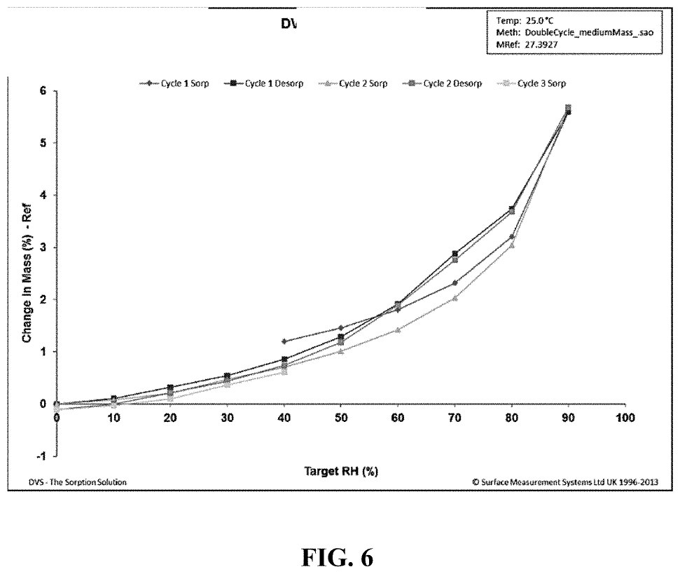

Gravimetric Vapor Sorption (GVS) shows an uptake of 6% between 0% and 90% RH. The sample is hygroscopic. The GVS isotherm plot is provided in FIG. 6.

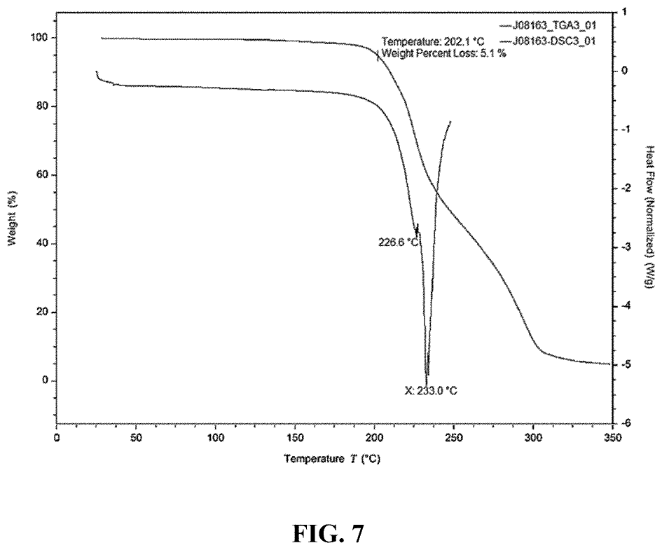

The combined DSC/TGA results for (R)-2-(2-hydroxyphenylamino)-5,5-dimethyl-4,5-dihydrothiazole-4-carboxylic acid mono-hydrochloride is provided in FIG. 7. The DSC shows a split endotherm between 200° C. and 250° C. and the TGA shows that decomposition (total 5% mass loss) starts at ˜202° C.

### Example 3: Method of Manufacturing Compound 1

A method of making a compound of Formula I (Compound 1) is provided.

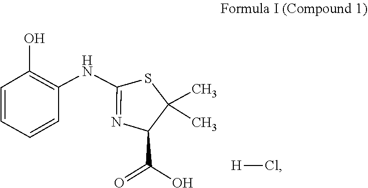 [SMILES: CC1(C)SC(Nc2ccccc2O)=N[C@@H]1C(=O)O ; Cl]

The method includes reacting an amine with a structure of

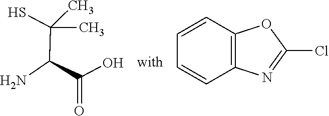

in the presence of a base and a first solvent to form an intermediate product of Formula II:

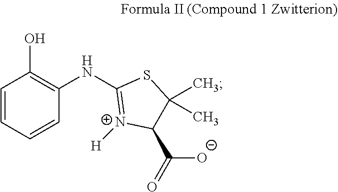 [SMILES: CC1(C)SC(Nc2ccccc2O)=[NH+][C@@H]1C(=O)[O-]]

and contacting the intermediate product with an acid and a second solvent to form Compound 1. In various embodiments, Compound 1 Zwitterion is isolated prior to being to being treated with acid.

The base can be any suitable base such as, without limitation, a primary, secondary, or tertiary amine, an alkyl lithium, a Grignard reagent, or an alkali metal hydroxide. In various embodiments, the base is selected from the group consisting of LiOH, NaOH, KOH, and combinations thereof. In various embodiments, the base is NaOH.

The first solvent can be any suitable solvent that is capable of dissolving the starting materials. The first solvent can be, in various embodiments, a polar protic solvent, a polar aprotic solvent, or a combination thereof. Suitable polar protic solvents can be, in various embodiments, water, methanol, ethanol, trifluoroethanol, iso-propanol, and mixtures thereof. In various embodiments, the polar aprotic solvent can be acetone, tetrahydrofuran, dimethylsulfoxide, acetonitrile, N,N-dimethylformamide, N-methyl-2-pyrrolidone, and mixtures thereof. The first solvent can also be a mixture of a protic polar solvent and an aprotic polar solvent, in any suitable ratio, such as from about 1:1 (protic:aprotic) to about 1:10 (protic:aprotic), or about 10:1 (protic:aprotic). In various embodiments, the first solvent is water.

The acid can be any suitable inorganic acid, such as HF, HCl, HBr, H₂SO₄, HNO₃, H₃NSO₃, H₃PO₄, and the like. The acid can also be an organic acid, such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like. In various embodiments, the acid is hydrochloric acid (HCl).

The second solvent can be any suitable solvent that is capable of dissolving polar substances such as Compound 1 Zwitterion. The second solvent can be, in various embodiments, a polar protic solvent, a polar aprotic solvent, or a combination thereof. Suitable polar protic solvents can be, in various embodiments, water, methanol, ethanol, trifluoroethanol, iso-propanol, and mixtures thereof. In various embodiments, the polar aprotic solvent can be acetone, tetrahydrofuran, dimethylsulfoxide, acetonitrile, N,N-dimethylformamide, N-methyl-2-pyrrolidone, and mixtures thereof. The second solvent can also be a mixture of a protic polar solvent and an aprotic polar solvent, in any suitable ratio, such as from about 1:1 (protic:aprotic) to about 1:10 (protic:aprotic), or about 10:1 (protic:aprotic). In various embodiments, the second solvent is iso-propanol.

In various embodiments, Compound 1 can be prepared according to Scheme 1 as follows:

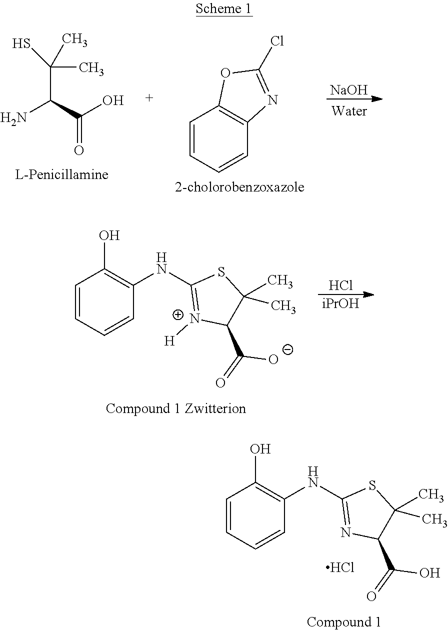

In various embodiments, Compound 1 Zwitterion can be prepared according to Scheme 2:

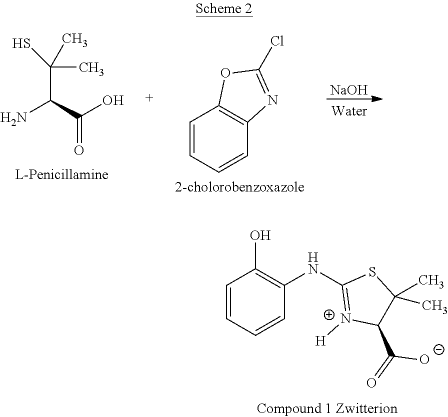

Purified water (8 volumes) is degassed with argon for approximately 30 minutes. L-penicillamine (1.6756 mol.) is added and stirred for approximately 10 minutes maintaining the temperature below 30° C. The mixture is cooled to 10±5° C. A cooled solution of sodium hydroxide (3.3512 mol.) in degassed water (2 volumes) is added slowly to the above mass while maintaining temperature below 20° C., followed by slow addition of 2-chlorobenzoxazole (1.8431 mol) below 30° C. After complete addition the reaction mass is allowed to reach ambient temperature and is stirred for not less than 8 h at ambient temperature. Upon completion of the reaction, the reaction mixture is cooled to 10±5° C., diluted with isopropyl alcohol (10 volumes) and acidified to pH 4.3-4.6 by dropwise addition of 2N aqueous hydrochloric acid below 30° C. The solution is stirred for approximately 16 hours at below 5±5° C. The solid is isolated by filtration, washed with isopropyl alcohol (3 volumes), and dried to get the zwitterion as white solid.

In various embodiments, Compound 1 can be prepared according to Scheme 3:

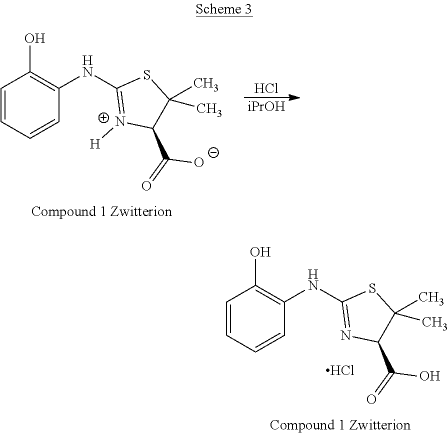 [SMILES: "iPrOH" ; CC1(C)SC(Nc2ccccc2O)=[NH+][C@@H]1C(=O)[O-] ; Cl ; CC1(C)SC(Nc2ccccc2O)=N[C@@H]1C(=O)O ; Cl]

The zwitterion is added to isopropyl alcohol (17.5 volumes) and cooled to 5±5° C. Freshly prepared 2M HCl in isopropyl alcohol (1.05 equivalence with regard to zwitterion) is added below 10° C. The mixture is stirred for approximately 15 min, and the clear solution filtered under inert atmosphere. The filtrate is stirred not less than 16 h at 5±5° C. The mixture is concentrated to approximately 3 volumes below 30° C., methyl tertiary-butyl ether (MTBE) is added (5 volumes) and kept at 5±5° C. for not less than 20 h. The solid formed is isolated by filtration and washed with MTBE (3 volumes). The isolated solid is dried in vacuum tray drier at 50±5° C. for approximately 12 h to obtain Compound 1 as white solid.

des-HCl Compound 1 (i.e. lacking the HCl addition salt of Compound 1) can be prepared according to Scheme 4:

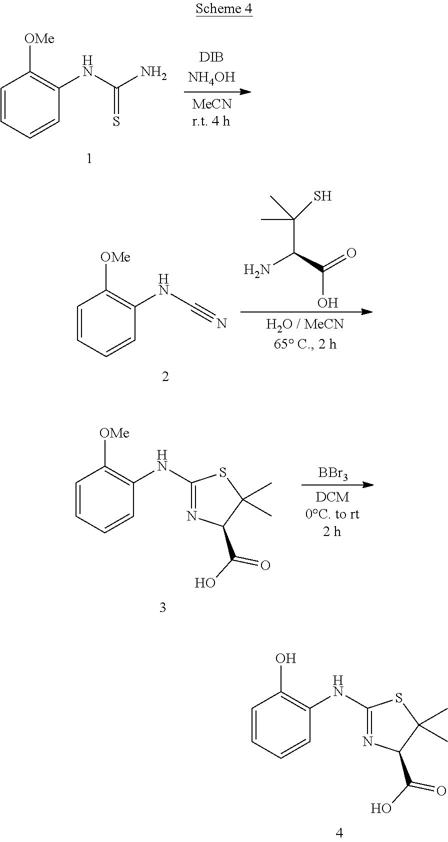

(i) Preparation of N-(2-Methoxyphenyl)cyanamide (2):

Aqueous ammonia (25%, 90 mL) was added to a stirred and ice-cooled suspension of 1-(2-methoxyphenyl)thiourea (1) (5.00 g, 27.44 mmol) in acetonitrile (90 mL). Diacetoxyiodobenzene (10.60 g, 32.92 mmol) was added portion-wise over a period of 10 min. The reaction mixture was stirred at room temperature for 4 h, and the precipitated sulfur was filtered. The filtrate was concentrated (until ½ of volume) and extracted with ethyl acetate (3×20 mL). The ethyl acetate layer was washed with water (2×30 mL) and then with brine (50 mL). The organic layer was dried over anhydrous solid Na₂SO₄, filtered and the filtrate concentrated under reduced pressure. The resultant residue was purified by flash column chromatography using petroleum ether/ethyl ether (1:1) to give the N-(2-methoxyphenyl)-cyanamide (2) (3.33 g, 82% yield). 300 MHz ¹H-NMR (CDCl₃, ppm): 7.08 (ddd, J=7.5, 1.9, 0.5 Hz, 1H) 7.04 (ddd, J=7.5, 7.5, 1.9 Hz) 6.98 (ddd, J=7.5, 7.5, 1.7 Hz) 6.88 (dd, J=7.5, 1.7 Hz) 6.26 (s, 1H) 3.88 (s, 3H). ESI-MS (m/z): 149 [M+H]⁺.

(ii) Preparation of ((R)-2-((2-methoxyphenyl)amino)-5,5-dimethyl-4,5-dihydrothiazole-4-carboxylic acid (3)

A mixture N-(2-methoxyphenyl)cyanamide (2) (1.00 g, 6.75 mmol) and L-penicillamine (1.21 g, 8.10 mmol) in deionized water/acetonitrile (20 mL/20 mL) was heated at reflux under an argon atmosphere for 2 h. The mixture was then concentrated under reduced pressure, and residue purified by reverse phase chromatography to afford (R)-2-((2-methoxyphenyl)amino)-5,5-dimethyl-4,5-dihydrothiazole-4-carboxylic acid (3) (0.92 g, 49% yield). 300 MHz ¹H-NMR (CD₃OD, ppm): 7.43-7.33 (m, 2H) 7.15 (dd, J=8.3, 1.1 Hz, 1H) 7.03 (ddd, J=7.7, 7.7, 1.2 Hz) 4.42 (s, 1H) 3.91 (s, 3H) 1.77 (s, 3H) 1.60 (s, 3H). ESI-MS (m/z): 281 [M+H]⁺

### (iii) Preparation of (R)-2-((2-hydroxyphenyl)amino)-5,5-dimethyl-4,5-dihydrothiazole-4-carboxylic acid (4)

Neat BBr₃ (2.19 mL, 12.84 mmol) was added to a solution of ((R)-2-((2-methoxyphenyl)amino)-5,5-dimethyl-4,5-dihydrothiazole-4-carboxylic acid (3) (360 mg, 1.28 mmol) in CH₂Cl₂ (20 mL) at 0° C. temperature. The reaction mixture was stirred at ambient temperature for 3 h, then water (2 mL) was added and the resulting suspension was stirred for 10 min. The resultant precipitate was filtered and removed. The filtrate was evaporated and purified by reverse phase chromatography to afford (R)-2-((2-hydroxyphenyl)amino)-5,5-dimethyl-4,5-dihydrothiazole-4-carboxylic acid (4) (210 mg, 64% yield). 300 MHz ¹H-NMR (CD₃OD, ppm): 6.94-6.86 (m, 2H) 6.82-6.77 (m, 1H) 6.73 (ddd, J=7.5, 7.5, 1.5 Hz) 4.19 (s, 1H) 3.91-1.68 (s, 3H) 1.49 (s, 3H). ESI-MS (m/z): 267 [M+H]⁺

Although Compound 1 is a hydrochloride acid addition salt of 4, other pharmaceutically acceptable acid addition salts of 4 can be used in the methods described herein. Pharmaceutically-acceptable acids refers to those acids that are not toxic or otherwise biologically undesirable. Pharmaceutically acceptable acid addition salts can be formed with pharmaceutically-acceptable inorganic acids including, but not limited to, hydrobromic acid, sulfuric acid, sulfamic acid, nitric acid, phosphoric acid, and the like.

Pharmaceutically acceptable acid addition salts can also be formed with pharmaceutically acceptable organic acids. Examples of pharmaceutically-acceptable organic acids, include but are not limited to, acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

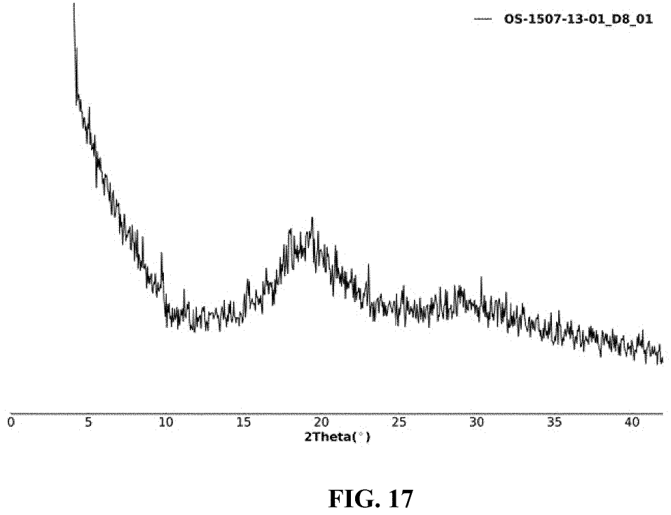

An amorphous form of Compound 1 can also be made by, for example, lyophilizing (crystalline) Compound 1 as follows: (R)-2-(2-hydroxyphenylamino)-5,5-dimethyl-4,5-dihydrothiazole-4-carboxylic acid mono-hydrochloride (Compound 1, 200 mg) was dissolved in tert-butanol:water system (1:1 ratio, 40 vol., 8 ml) at RT. The solution was filtered to remove potential seeds and the filtered solution was frozen in a round bottom flask over a bath of dry ice and acetone. The sample was then set for freeze-drying. The recovered solid was analyzed by the appropriate techniques. The XPRD spectrum of amorphous Compound 1 is shown in FIG. 17.

### Example 4: Manufacturing Controls

Stage 1

Stage 1 in-process controls include testing for reaction completion (percent-unreacted L-penicillamine), residual solvents (including triethylamine), moisture content and assay/related substances after initial drying and loss on drying and residue on ignition after final drying.

Stage 2

Stage 2 in-process controls include titration of isopropanol/HCl addition, testing of crude reaction mixture for assay/related substances, chiral impurity, L-penicillamine and residue on ignition (ROI). After purification, ROI testing is repeated. After drying residual solvents and moisture is determined.

### Example 5: Control of Materials

### 1. Starting Materials

(L)-Penicillamine and 2-chlorobenzoxazole have been selected as the regulatory starting materials for clinical material produced to support Phase I clinical studies. The structures are provided in Table 5.

TABLE 5
Structures of Regulatory Starting Materials

| Starting Material | Chemical Structure |
|---|---|
| (L)-Penicillamine | 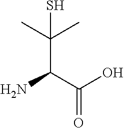 |
| 2-Chlorobenzoxazole | 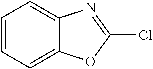 |

The starting materials are commercially available and are tested to ensure that acceptance criteria are met prior to use. The release specifications for (L)-penicillamine and 2-chlorobenzoxazole are provided in Table 6 and Table 7, respectively.

TABLE 6
(L)-Penicillamine Release Specifications

| Test Attribute | Release Specification |
|---|---|
| Appearance | Off-white to white solid |
| Identification by ¹H-NMR, IR, and Mass | Complies with the structure |
| Spectroscopy | |
| Chromatographic Purity by HPLC (ELSD) | NLT ^{a} 98.5% |
| Total Impurities | NMT ^{b} 1.5% |
| Dimer | NMT 1.0% |
| Chiral Purity by HPLC | NLT 99.0% |
| Loss on Drying | NMT 1.0% |

^{a} NLT = not less than
^{b} NMT = not more than

TABLE 7
2-Chlorobenzoxazole Release Specifications

| Test Attribute | Release Specification |
|---|---|
| Appearance | Colorless to pale yellow liquid |
| Identification by 1H-NMR | Complies with structure |
| Purity (area %) by GC | NLT ^{a} 98.0% |
| BO-Imp-1 | NMT ^{b} 1.0% |

^{a} NLT = not less than
^{b} NMT = not more than

### Example 6: Analytical Testing of Batches of Compound 1

Batches of Compound 1 suitable for administration to individuals and prepared according to the method describe herein were analyzed for purity. The structures of the impurities (Imp designations in Tables 8A-8B), are depicted in Table 10.

TABLE 8A
In-Process Testing for Compound 1

| | | | |
|---|---|---|---|
| Step | Test | Method | Action Limit |

Step 1: Preparation of Compound 1 Zwitterion

| | | | |
|---|---|---|---|
| After Initial | % L-Penicillamine | HPLC ELSD | NMT ^{a} 1.0% |
| Reaction | | | |
| After Initial | Water Content | Karl Fischer | NMT 1.0% |
| Drying | | | |
| | Purity and Related | HPLC UV | Zwitterion: NLT ^{b} 98.5% |
| | Substances of | | 2-Cl BO: NMT 0.15% |
| | Zwitterion | | BO-Imp-1: NMT 0.15% |
| | | | BO-Imp-2: NMT 0.15% |
| | | | BO-Imp-3: NMT 0.15% |
| | | | BO-Imp-4: NMT 0.15% |
| | | | BO-Imp-5: NMT 0.15% |
| | | | Compound 1-Imp-3: NMT 0.5% |
| | % L-Penicillamine | HPLC ELSD | NMT 0.3% |
| | Chiral Impurity | HPLC UV | NMT 1.0% |
| | Benzene | GC | NMT 2 ppm |
| | Triethylamine | GC-MS | NMT 320 ppm |
| After Final Drying | Loss on Drying | USP <731> | Report result |
| | Residue on Ignition | USP <281> | Report result |

Step 2: Preparation of Compound 1

| | | | |
|---|---|---|---|
| Isopropyl | Molarity | Titration | Report result |
| Alcohol/HCL | | | |
| After Initial | Purity and Related | HPLC UV | Purity: NLT 98.5% |
| Reaction | Substances | | 2-Cl BO: NMT 0.15% |
| | | | BO-Imp-1: NMT 0.15% |
| | | | BO-Imp-2: NMT 0.15% |
| | | | BO-Imp-3: NMT 0.15% |
| | | | BO-Imp-4: NMT 0.15% |
| | | | BO-Imp-5: NMT 0.15% |

TABLE 8B
In-Process Testing for Compound 1

| Step | Test | Method | Action Limit |
|---|---|---|---|
| | | | Compound 1-Imp-3: NMT 0.5% |
| | | | Unspecified Impurities: |
| | | | NMT 0.15% |
| | Chiral Impurity | HPLC UV | NMT 1.0% |
| | % L-Penicillamine | HPLC ELSD | NMT 0.5% |
| | Residue on Ignition | ROI | Report results |
| After Purification | Residue on Ignition | ROI | NMT 0.25% |
| After Drying | Residual Solvents | GC | Ethanol: NMT 5,000 ppm |
| | | | n-Butanol: NMT 5,000 ppm |
| | | | Isopropyl alcohol: |
| | | | NMT 5,000 ppm |
| | | | Methyl tert-butyl ether: |
| | | | NMT 5,000 ppm |
| | | | Chloroform: NMT 60 ppm |
| | | | 1,2-Dichloroethane: |
| | | | NMT 5 ppm |
| | Water | Karl Fischer | NMT 1.0% |

^{a} NMT = not more than
^{b} NLT = not less than

### Example 7: Analytical Methods

Analytical methods, in various embodiments, were carried out with equipment and parameters set forth below. The testing was conducted on batches Compound 1 suitable for administration to individuals according to the methods and specifications belonging to the USP (United States Pharmacopeia).

TABLE 9
Analytical Procedures For Compound 1

| Test | Summary of the Analytical Procedure | | | |
|---|---|---|---|---|
| Description | Visual Examination | | | |
| IR Identification | FT-IR | | | |
| HPLC Method 1 | Identification is confirmed by verifying the retention time of the Compound 1 | | | |
| Identification, | peak in the drug substance is consistent with that of the working standard. | | | |
| Purity, Assay, | Purity, assay, and related substances are performed using reversed-phase | | | |
| and Impurities | HPLC and the following chromatographic conditions. | | | |
| BO-Imp⁻1, | Instrument | Suitable HPLC with variable wavelength UV | | |
| BO-Imp-4, | | detector | | |
| BO-Imp-5, | Column | X-Bridge C18, 250 × 4.6 mm, 5 μm | | |
| Compound 1 Imp-3, | Mobile Phase A | 25 mM K₂HPO₄ in water, pH 8.4:Methanol (95:5) | | |
| Individual | Mobile Phase B | Acetonitrile:Methanol (50:50) | | |
| Unspecified | | | | |
| Impurities, | | | | |
| Total Impurities | | | | |
| | | | % Mobile | % |
| | | Time | Phase A | Mobile |
| | Gradient | 0.01 | 75 | 25 |
| | | 2.00 | 75 | 25 |
| | | 12.00 | 55 | 45 |
| | | 18.00 | 55 | 45 |
| | | 35.00 | 35 | 65 |
| | | 40.00 | 35 | 65 |
| | | 40.10 | 75 | 25 |
| | | 50.00 | 75 | 25 |
| | Flow Rate | 1.0 mL/min | | |
| | Injection Volume | 8.0 μL | | |
| | Wavelength | 225 nm | | |
| | Column | 30° C. | | |
| | Temperatur | | | |
| | Detector | 40° C. | | |
| | Cell | | | |
| | Run Time | 50 minutes | | |
| Test | Summary of the Analytical Procedure | | | |
| HPLC Method 2 | A limit test is performed for process impurities 2-Cl—BO and BO-Imp-2 are | | | |
| Limit Test 2-Cl—BO | performed using reversed-phase HPLC and the following chromatographic | | | |
| and BO-Imp-2 | conditions. | | | |
| | Instrument | Suitable HPLC with variable wavelength UV | | |
| | | detector | | |
| | Column | X-Bridge C18, 250 × 4.6 mm, 5 μm | | |
| | Mobile Phase A | 25 mM K₂HPO₄ in water, pH 8.4:Methanol (95:5) | | |
| | Mobile Phase B | Acetonitrile:Methanol (50:50) | | |
| | | | % Mobile | % |
| | | Time | Phase A | Mobile |
| | Gradient | 0.01 | 75 | 25 |
| | | 2 | 75 | 25 |
| | | 12 | 55 | 45 |
| | | 18 | 55 | 45 |
| | | 35 | 35 | 65 |
| | | 40 | 35 | 65 |
| | | 40.1 | 75 | 25 |
| | | 50 | 75 | 25 |
| | Flow Rate | 1.0 mL/min | | |
| | Injection Volume | 10.0 μL | | |
| | Wavelength | 250 nm | | |
| | Column | 30° C. | | |
| | Temperatur | | | |
| | Detector | 40° C. | | |
| | Cell | | | |
| | Run Time | 50 minutes | | |
| Test | Summary of the Analytical Procedure | | | |
| HPLC Method | A limit test for BO-Imp-3 is performed using reversed-phase HPLC and the | | | |
| 3 BO-Imp-3 | following chromatographic conditions. | | | |
| | Instrument | Suitable HPLC with variable wavelength UV | | |
| | | detector | | |
| | Column | Waters X-Bridge C18, 250 × 4.6 mm, 5 μm | | |
| | Mobile Phase A | 25 mM K₂HPO₄ in water, pH 8.4:methanol (95:5) | | |
| | Mobile Phase B | Acetonitrile:Methanol (50:50) | | |
| | | | % | % |
| | | Time | Mobile | Mobile |
| | Gradient | 0.01 | 75 | 25 |
| | | 2 | 75 | 25 |
| | | 12 | 55 | 45 |
| | | 18 | 55 | 45 |
| | | 35 | 35 | 65 |
| | | 40 | 35 | 65 |
| | | 40.1 | 75 | 25 |
| | | 50 | 75 | 25 |
| | Flow Rate | 1.0 mL/min | | |
| | Injection Volume | 10 μL | | |
| | Wavelength | 225 nm | | |
| | Column | 30° C. | | |
| | Temperatur | | | |
| | Autosampler | 15° C. | | |
| | Detector | 40° C. | | |
| | Cell | | | |
| | Run Time | 50 minutes | | |
| HPLC Method 4 | A limit test for L-penicillamine is performed using reversed-phase HPLC | | | |
| (L)- | using a MS detector and the following chromatographic conditions. | | | |
| Penicillamine | | | | |
| Test | Summary of the Analytical Procedure | | | |
| | | | % | % |
| | | Time | Mobile | Mobile |
| | Gradient | 0.0 | 100 | 0 |
| | | 10 | 20 | 80 |
| | | 16 | 20 | 80 |
| | | 17 | 100 | 0 |
| | | 22 | 100 | 0 |
| | Flow Rate | 0.5 mL/min | | |
| | Injection Volume | 10 μL | | |
| | Wavelength | 254 nm | | |
| | Column | 35° C. | | |
| | Temperature | | | |
| | Run Time | 22 minutes | | |
| | Mass Parameters | | | |
| | Nebulizer Pressure | 40 psi | | |
| | Dry Gas Flow Rate | 10 L/min | | |
| | Fragmentor Voltage | 70 V | | |
| | Capillary Voltage | 3,000 V | | |
| | Dry Gas | 350° C. | | |
| | Temperature | | | |
| | Collection Mode | SIM mode: positive signal for 150 ion | | |
| HPLC Method 5 | A limit test for BO-Imp-3 is performed using chiral HPLC and the following | | | |
| S-Compound 1 Imp- | chromatographic conditions. | | | |
| 3 | Instrument | Suitable HPLC with variable wavelength UV | | |
| | | detector | | |
| | Column | Chiralpak IG, 250 × 4.6 mm, 5 μm | | |
| | Mobile Phase: | 0.1% diethylamine in acetonitrile:methanol 95:5 | | |
| | Flow Rate | 0.8 mL/min | | |
| | Injection Volume | 10 μL | | |
| | Wavelength | 285 nm | | |
| | Column | 25° C. | | |
| | Temperature | | | |
| | Autosampler | 25° C. | | |
| | Temperature | | | |
| | Detector Cell | 40° C. | | |
| | Temperature | | | |
| | Run Time | 70 minutes | | |
| Test | Summary of the Analytical Procedure | | | |
| HPLC Method | Quantitation of (S)-Compound 1 is performed using chiral HPLC | | | |
| 6 Chiral Purity | chromatography and the following chromatographic conditions. | | | |
| | Instrument | Suitable HPLC with variable wavelength detector | | |
| | Column | Chiralcel OX-3, 250 × 4.6 mm, 3 μm | | |
| | Mobile Phase A | 0.3% trifluoroacetic acid in n-hexane | | |
| | Mobile Phase B | 0.1% diethylamine in ethanol:isopropyl alcohol 8:2 | | |
| | | | % Mobile | % Mobile |
| | | Time | Phase A | Phase B |
| | Gradient | 0.01 | 80 | 20 |
| | | 15.0 | 80 | 20 |
| | Flow Rate | 1.0 mL/min | | |
| | Injection Volume | 10 μL | | |
| | Wavelength | 285 nm | | |
| | Column | 25° C. | | |
| | Temperature | | | |
| | Detector Cell | 40° C. | | |
| | Temperature | | | |
| | Run Time | 15 minutes | | |
| Residual Solvents | Quantitation of ethanol, isopropyl alcohol, n-butanol, and methyl tert-butyl | | | |
| Ethanol, Isopropyl | ether is performed using a headspace GC method and flame ionization | | | |
| Alcohol, n- | detection. The chromatographic conditions are listed below. | | | |
| Butanol, MTBE^{a} | Instrument | Suitable GC with flame ionization detector (FID) | | |
| | Column | DB-1, 60 m × 0.32 mm, 3 μm | | |
| | Carrier Gas | Helium | | |
| | | Rate | Temperature | Hold Time |
| | | (° C./min) | (° C.) | (Minutes) |
| | Temperature | — | 50 | 2 |
| | Program | 3 | 80 | 5 |
| | | 15 | 260 | 11 |

| | Flow Rate | 1.5 mL/min |
|---|---|---|
| | Injection Mode | Split |
| | Split Ratio | 10:1 |
| | Detector | 280° C. |
| | Temperature | |
| | Make-Up Gas | Helium |
| | Make-Up Flow | 30.0 mL/min |
| | H2 Flow | 40.0 mL/min |
| Test | Summary of the Analytical Procedure | |
| | Air Flow | 400.0 mL/min |
| | Run Time | 40.0 minutes |
| Residual | Quantitation of chloroform is performed using a GC method and electron | |
| Solvent | impact mass detection. The chromatographic conditions are listed below. | |
| Chloroform | Instrument | Suitable GC with electron impact mass detection |
| | Column | DB-1, 60 m × 0.32 mm, 3 μm |
| | Carrier Gas | Helium |
| | Oven Temperature | 50° C., hold at ° C. for 2 minutes |
| | Temperature Ramp | 50° C. to 80° C. at 3° C./min, hold at 80° C. for 7 minutes |
| | | 80° C. to 260° C. at 50° C./min, hold at 260° C. for |
| | | 12 minutes |
| | Flow Rate | 1.0 mL/min |
| | Injection Mode | Split |
| | Split Ratio | 10:1 |
| | Injector | 200° C. |
| | Temperature | |
| | Injection Volume | 2 μL |
| | Make-Up Flow | 30.0 mL/min |
| | Run Time | 34.6 minutes |
| Residual Solvent | Quantitation of 1,2-Dichloroethane is performed using a GC method and | |
| 1,2- | electron impact mass detection. The chromatographic conditions are listed | |
| Dichloroethane | below. | |
| | Instrument | Suitable GC with electron impact mass detection |
| | Column | DB-624, 30 m × 0.32 mm, 1.8 μm |
| | Carrier Gas | Helium |
| | Oven Temperature | 40° C., hold at 40° C. for 5 minutes |
| | Temperature Ramp | 40° C. to 60° C. at 4° C./min, hold at 60° C. for 1 minute |
| | | 60° C. to 250° C. at 50° C./min, hold at 250° C. for |
| | | 6 minutes |
| | Flow Rate | 1.5 mL/min |
| | Injection Mode | Split |
| | Split Ratio | 5:1 |
| | Injector Temperature | 220° C. |
| | Injection Volume | 1 μL |
| | Run Time | 20.8 minutes |

| Water | USP <921>, Method Ia |
|---|---|
| Residue on Ignition | USP <281> |
| Test | Summary of the Analytical Procedure |
| Elemental | Arsenic (As), cadmium (Cd), mercury (Hg), lead (Pb), cobalt (Co), |
| Impurities Arsenic, | vanadium (V), and nickel (Ni) content are determined using Inductively |
| Cadmium, | Coupled Plasma (ICP) with mass spectral detection. |
| Mercury, Lead, | |
| Cobalt, Vanadium, | |
| and Nickel | |
| Elemental | Lithium (Li), antimony (Sb), and copper (Cu), content are determined |
| Impurities | using ICP with Optical Emission Spectroscopy (OES) detection. |
| Lithium, | |
| Powder XRD | USP <941> |
| Microbial Analysis | USP <61>, USP <62> |

### Example 8: Impurities in Clinical Batches of Compound 1

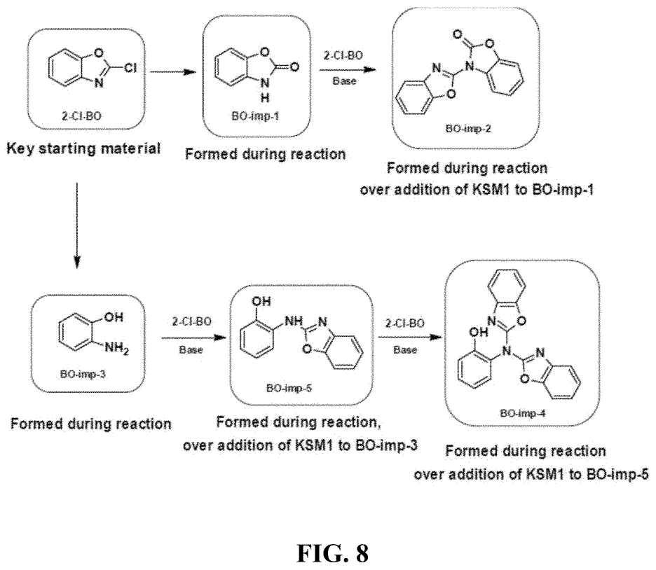

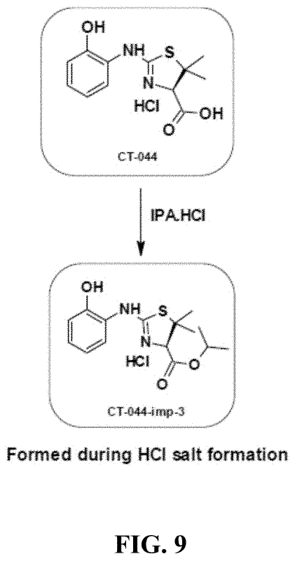

In various embodiments, any of the compositions containing Compound 1 can contain up to 0.15% of one or more impurities set forth in Table 10 below, and as shown in FIG. 8 and FIG. 9:

TABLE 10
Impurities Identified in Batches of Compound 1

| Abbreviation | Chemical Name | Structure |
|---|---|---|
| 2-Cl—BO | 2-Chlorobenzoxazole | 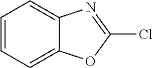 |
| L-Penicillamine | L-Penicillamine | 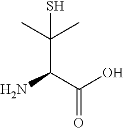 |
| BO-Imp-1 | 2-Hydroxybenzoxazole | 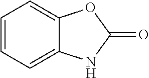 |
| BO-Imp-2 | 2′H-[2,3′-bi-1,3- benzoxazol]-2′-one | 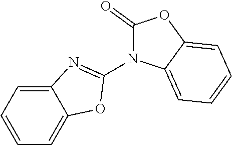 [SMILES: O=c1oc2ccccc2n1-c1nc2ccccc2o1] |
| BO-Imp-3 | 2-Aminophenol | 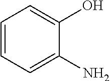 |
| BO-Imp-4 | 2-[Bis(1,3-benzoxazol-2-y1) amino]phenyl | 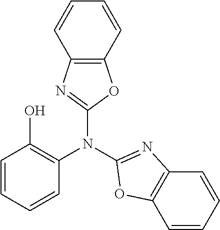 [SMILES: Oc1ccccc1N(c1nc2ccccc2o1)c1nc2ccccc2o1] |
| BO-Imp-5 | 2[(1,3-Benzoxazol-2- yl)amino]phenol | 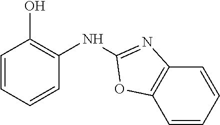 [SMILES: Oc1ccccc1Nc1nc2ccccc2o1] |
| Cmp1 Imp-3 | Propan-2-y1(4R)-2-(2- hydroxyanilino)-5,5-dimethyl-4,5- dihydro-1,3-thiazole-4-carboxylate | 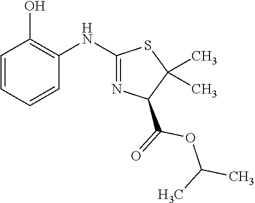 [SMILES: CC(C)OC(=O)[C@H]1N=C(Nc2ccccc2O)SC1(C)C] |

In various embodiments, the composition including the therapeutically effective amount of Compound 1 has less than about 0.30% w/w, 0.25% w/w, 0.20% w/w, or 0.15% w/w of at least one impurity selected from the group consisting of 2-Cl-BO, BO-Imp-1, BO-Imp-2, BO-Imp-3, BO-Imp-4, BO-Imp-5, and Cmp1 Imp-3. In various embodiments, the composition including the therapeutically effective amount of Compound 1 has about 0.0001% to about 0.30% w/w, about 0.0001% to about 0.25% w/w, about 0.0001% to about 0.20% w/w, about 0.001% to about 0.15% w/w, or about 0.01% to about 0.15% w/w of at least one impurity selected from the group consisting of 2-Cl-BO, BO-Imp-1, BO-Imp-2, BO-Imp-3, BO-Imp-4, BO-Imp-5, and Cmp1 Imp-3. In various embodiments, the composition including the therapeutically effective amount of Compound 1 has about 0.0005%, 0.001%, 0.002%, 0.003%, 0.004%, 0.005%, 0.006%, 0.007%, 0.008%, 0.009%, 0.010%, 0.012%, 0.014%, 0.016%, 0.018%, 0.020%, 0.022%, 0.024%, 0.026%, 0.028%, 0.030%, 0.032%, 0.034%, 0.036%, 0.038%, 0.040%, 0.042%, 0.044%, 0.046%, 0.048%, or 0.050% w/w of at least one impurity selected from the group consisting of 2-Cl-BO, BO-Imp-1, BO-Imp-2, BO-Imp-3, BO-Imp-4, BO-Imp-5, and Cmp1 Imp-3. In various embodiments, the composition including the therapeutically effective amount of Compound 1 includes about 0.010% to about 0.020% w/w of impurity BO-Imp-1 and about 0.002% to about 0.004% w/w of impurity BO-Imp-5.

Impurities BO-Imp-1 through BO-Imp-5 can arise from the 2-chlorobenzoxazole starting material. A flow chart showing the formation of these impurities is provided in FIG. 8.

BO-Imp-3 is a process impurity which forms by hydrolysis of 2-chlorobenzoxazole by a minor competitive reaction pathway with sodium hydroxide. It is purged by filtration of the zwitterion of Compound 1. BO-Imp-3 forms as a minor impurity (0.3%) during forced degradation studies only at the most-harsh conditions of 5N sodium hydroxide for 5 h and is unlikely to be a degradant under the recommended or accelerated drug substance storage conditions.

Cmp1 Imp-3 is a process impurity that forms via acid catalyzed esterification of salt-free Compound 1 with isopropanol solvent during the hydrochloride salt formation. Its formation is minimized by using stoichiometric hydrogen chloride in isopropanol, which is added to a pre-cooled suspension of the zwitterion in isopropanol. It is purged by filtration of Compound 1. Cmp1 Imp-3 is formed as shown in FIG. 9.

In various embodiments, the manufacture of Compound 1 produces the following impurities as set forth in Table 11:

TABLE 11
Compound 1 Drug Substance Specifications

| | | Specification (Acceptance |
|---|---|---|
| Parameter | Test Method | Criteria Applied) |
| Description | Visual | White to off-white solid |
| | Examination | |
| Identification | | |
| IR | FT-IR | Conforms to structure |
| HPLC | HPLC Method 1 | The retention time of the |
| | | principal peak in |
| | | the sample chromatogram cor- |
| | | responds to that |
| | | of the standard chromatogram |
| Chloride | USP<191> | With Silver Nitrate TS, solu- |
| | Test A | tion of chlorides |
| | | yield a white, curdy precip- |
| | | itate that is |
| | | insoluble in nitric acid but |
| | | is soluble in a |
| | | slight excess of 6N |
| | | ammonium hydroxide |
| Purity | HPLC Method 1 | NLT ^{a} 98.5% (% area) |
| Assay | HPLC Method 1 | 97.0%-103.0% |
| Impurities | | |
| 2-Cl—BO | HPLC Method 2 | NMT 0.004% |
| BO-Imp-1 | HPLC Method 1 | NMT 0.15% |
| BO-Imp-2 | HPLC Method 2 | NMT 0.004% |
| BO-Imp-3 (2- | HPLC Method 3 | NMT 0.004% |
| aminophenol) | | |
| BO-Imp-4 | HPLC Method 1 | NMT 0.15% |
| BO-Imp-5 | | NMT 0.15% |
| Compound 1 Imp-3 | | NMT 0.5% |
| L-Penicillamine | HPLC Method 4 | NMT 0.004% |
| S-Compound 1 Imp-3 | HPLC Method 5 | NMT 0.15% |
| Chiral Purity | HPLC Method 6 | NMT^{b} 0.5% S-Isomer |
| Any Individual | HPLC Method 1 | NMT 0.15% |
| Unspecified Impurity | | |
| Total Impurities | | NMT 1.5% |
| Residual Solvents | | |
| Ethanol | GC-HS Method 1 | NMT 5,000 ppm |
| Isopropyl Alcohol | | NMT 5,000 ppm |
| n-Butanol | | NMT 5,000 ppm |
| Methyl tert- | | NMT 5,000 ppm |
| butyl Ether | | |
| Chloroform | GC-MS Method 2 | NMT 60 ppm |
| 1,2-Dichloroethane | GC-MS Method 3 | NMT 5 ppm |
| Water | Karl Fischer | NMT 1.0% (w/w) |
| Residue on | USP <281> | NMT 0.25% w/w |
| Ignition | | |
| Elemental | | |
| Impurities | | |
| Arsenic | ICP-MS | NMT 1.5 ppm |
| Cadmium | | NMT 0.2 ppm |
| Mercury | | NMT 0.3 ppm |
| Lead | | NMT 0.5 ppm |
| Cobalt | | NMT 0.5 ppm |
| Vanadium | | NMT 1 ppm |
| Nickel | | NMT 2 ppm |
| Lithium | ICP-OES | NMT 55 ppm |
| Antimony | | NMT 120 ppm |
| Copper | | NMT 300 ppm |
| Powder XRD | XRPD | Crystalline |
| Microbial Analysis | | |
| TAMC | USP <61>, | NMT 10³ in 1 g |
| TYMC | USP <62> | NMT 10² in 1 g |
| E. coli | | Absent in 1 g |

In various embodiments, Compound 1 produced according to the methods described herein has the following analytical parameters:

TABLE 12
Data for Compound 1 Drug Substance Batches

| Batch Number | | | |
|---|---|---|---|
| Attribute | Proposed Specifications | Drug Batch | |
| Description | White to off-white solid | White solid | |
| Identification | | | |
| ¹H-NMR ^{a} | Conforms to structure | NT | |
| LC-MS ^{a} | Conforms to m/z | NT | |
| IR | IR spectrum conforms to the | Complies | |
| | structure of the molecule | | |
| HPLC | The retention time of the principal | Complies | |
| | peak in the sample chromatogram | | |
| | corresponds to that of the standard | | |
| | chromatogram | | |
| Chloride | With Silver Nitrate TS, solution of | Complies | |
| | chlorides yields a white, curdy | | |
| | precipitate that is insoluble in nitric | | |
| | acid but is soluble in a slight excess | | |
| | of 6N ammonium hydroxide | | |
| Purity | NLT 98.5% (% area) | 99.7 | |
| Assay | 97.0% 0 103.0% | 100.8 | |
| Specified Impurities | | | |
| 2-Cl—BO | NMT 0.004% | <0.004 ^{d} | |
| BO-Imp-1 | NMT 0.15% | 0.05 | |
| BO-Imp-2 | NMT 0.004% | <0.004 ^{d} | |
| BO-Imp-3 (2-aminophenol) | NMT 0.004% | <0.004 ^{d} | |
| BO-Imp-4 | NMT 0.15% | <0.013 | [LOD ^{e}) |
| BO-Imp-5 | NMT 0.15% | <0.045 | (LOQ ^{f}) |
| Compound 1 Imp-3 | NMT 0.5% | 0.16 | |
| L-Penicillamine | NMT 0.004% | <0.004 ^{d} | |
| S-Compound 1 Imp-3 | NMT 0.15% | <0.15 ^{d} | |
| Chiral Purity (S- | NMT 0.5% | <0.030 | (LOD) |
| Any Individual | NMT 0.15% | | |
| Unspecified | RRT 1.54 | <0.049 | (LOQ) |
| Impurity | RRT 1.85 | 0.11 | |
| | RRT 2.49 | <0.049 | (LOQ) |
| | RRT 3.27 | ND | |
| | RRT 3.87 | <0.049 | (LOQ) |
| | RRT 3.95 | <0.049 | (LOQ) |
| Total Impurities | NMT 1.5% | 0.3 | |
| Residual Solvents | | | |
| Ethanol | NMT 5,000 ppm | <150 | ppm (LOD) |
| Isopropyl Alcohol | NMT 5,000 ppm | 3,507 | ppm |
| n-Butanol | NMT 5,000 ppm | <150 | ppm (LOD) |
| MTBE | NMT 5,000 ppm | <150 | ppm (LOD) |
| Chloroform | NMT 60 ppm | <3.6 | ppm (LOD) |
| 1,2-Dichloroethane | NMT 5 ppm | <0.4 | ppm (LOD) |
| Water | NMT 1.0% (w/w) | 0.18 | |
| Residue on Ignition | NMT 0.25% w/w | 0.06 | |
| Elemental Impurities | | | |
| Arsenic | NMT 1.5 ppm | <0.225 | ppm (PDL) ^{g} |
| Cadmium | NMT 0.2 ppm | <0.03 | ppm (PDL) |
| Mercury | NMT 0.3 ppm | <0.045 | ppm (PDL) |
| Lead | NMT 0.5 ppm | <0.075 | ppm (PDL) |
| Cobalt | NMT 0.5 ppm | <0.15 | ppm (PDL) |
| Vanadium | NMT 1 ppm | <0.075 | ppm (PDL) |
| Nickel | NMT 2 ppm | <1.51 | ppm |
| Lithium | NMT 55 ppm | <3 | ppm |
| Antimony | NMT 120 ppm | <3 | ppm |
| Copper | NMT 300 ppm | <3 | ppm |

| | | |
|---|---|---|
| Powder XRD | Crystalline | Crystalline |
| Microbial Analysis | | |
| TAMC | NMT 10³ cfu in 1 g | <10 |
| TYMC | NMT 10² cfu in 1 g | <10 |
| E. coli | Absent in 1 g | Absent |

^{a} Testing performed for Batch A011800996 and is not required for routine release.
^{b} NT = not tested
c ND = not detected
^{d} Result obtained after development and qualification of Methods 2-5.
^{e} LOD = limit of detection
^{f} LOQ = limit of quantitation
^{g} PDL = practical detection limit

### Example 9: Pharmacology Overview

Compound 1 is a non-metal, orally bioavailable small molecule Reactive Species Decomposition Accelerant (RSDAx) which, in various embodiments, destroys peroxynitrite (PN) and/or hydrogen peroxide. Peroxynitrite and peroxide are powerful oxidants produced under conditions of injury and disease that cause untoward effects via protein nitration and modification of sensory ion channels leading to neuronal sensitization and pain.

In chemical-based assays of peroxynitrite (PN) oxidation, Compound 1 inhibits PN-mediated oxidation of small-molecule organic substrates such as luminol. In cell-based assays of PN-mediated cytotoxicity, Compound 1 is protective. Compound 1 can also catalytically remove peroxynitrite in models of protein nitration (a consequence of peroxynitrite oxidation) and in lactoperoxidase oxidation (mediated by peroxide) under physiological conditions (i.e., neutral pH). Chemically, Compound 1 can also react stoichiometrically with peroxynitrite to form a para-nitro adduct. Without being bound by theory, by targeting and removing peroxynitrite and peroxide, Compound 1 can disrupt the ensuing cascades that lead to hypersensitivity (protein modification, ion channel hyperexcitation) thus providing a long duration event in terms of pain relief.

In various embodiments, Compound 1 is efficacious in in vivo animal models of acute post-incisional hyperalgesia, both prophylactically and palliatively. Compound 1 alleviates allodynia in rat models of diabetic neuropathy (streptozotocin- and methylglyoxal-induced) without brain penetration, thereby avoiding common CNS side effects associated with gabapentin and duloxetine. In various embodiments, Compound 1 does not penetrate the blood-brain barrier (BBB). In various embodiments, less than about 1%, 0.8%, 0.6%, 0.4%, 0.2%, 0.1%, 0.08%, 0.06%, 0.04%, 0.02%, or 0.01% of Compound 1 in blood plasma penetrates the BBB. Compound 1 does not alter normal sensation when given to uninjured animals.

Compound 1 rapidly produces complete reversal of hypersensitivity caused by an injury/insult such as an incision or irritant and upon repeated dosing, reverses allodynia in models of painful diabetic neuropathy.

Compound 1 was examined in a variety of pharmacokinetic and metabolism studies. The compound was examined in detail in rat and dog, the species selected for toxicology studies. In vivo, no epimerization was found using chiral methods. The compound is bioavailable after oral administration with microgram amounts found in the plasma in both rat and dog. Female rats had higher exposure than males but exposure was similar between the sexes in dogs. In 28-day studies plasma concentrations reach Tₘₐₓ in 1 hour or less, and half-lives varied from approximately 3 to 8 hours in rat but were more consistent in dogs (t_{1/2}˜3.5 hours). Plasma concentrations in a 28-day pivotal rat and dog studies were very high, reaching Cₘₐₓ values of over 100 μg/mL at some doses.

Upon administration, Compound 1 is stable in both plasma and hepatocytes from rat, dog and human. Compound 1 is excreted into urine and feces of rats primarily as a sulfate conjugate. Compound 1 distributes to tissues but not to brain to an appreciable extent. Compound 1 is moderately protein-bound across species. Compound 1 does not inhibit major CYP isoforms (IC₅₀ for CYPs 3A4, 2D6, 1A2, 2C9, 2C19 are all >100 μM). Compound 1 does not inhibit P-gp, OATP1B1, OATP1B3 and OAT1, weakly inhibits OAT3 and modestly inhibits BCRP, which suggests that interactions with transporters or inhibition of CYPs would be minimal or absent at pharmacologically active doses.

### Example 10: Compound 1 Effects on Hyperalgesia in Rodent Incisional Models

Two incisional pain models were used to assess Compound 1 effects on hyperalgesia. The first model is referred to as the Brennan model in which, under anesthesia, a 1 cm longitudinal incision to the skin and underlying fascia of a rat hindpaw is made. The second is a variation but is a more invasive procedure in which the skin and muscle are spread apart using forceps which creates a longer-lasting hypersensitivity.

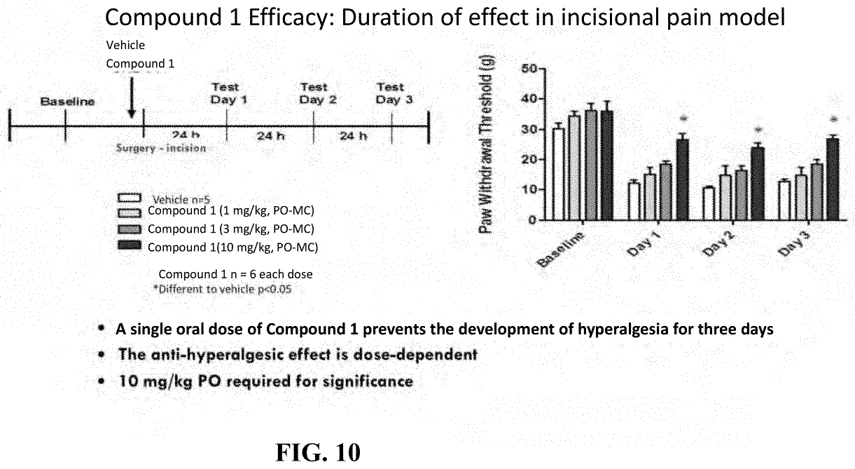

Using the Brennan protocol and a prophylactic paradigm, Compound 1 (3 doses given PO) or vehicle was administered 15 min prior to incision. At the 24-, 48-, and 72-h time points post-incision, mechanical thresholds were obtained (manual von Frey filament, using up/down method). Relative to the vehicle-treated cohort, animals receiving Compound 1 exhibited a reversal of hyperalgesia in a dose-dependent manner, as shown in FIG. 10.

The 10 mg/kg PO Compound 1 cohort showed a statistically significant reversal of hyperalgesia returning threshold values to nearly pre-injury baseline levels. In a subsequent experiment, the same paradigm was used with Compound 1 administered at a dose of 30 mg/kg PO along with positive control group consisting of celecoxib (30 mg/kg PO), morphine (10 mg/kg SC), and vehicle. At the 24-, 48-, and 72-h time points post-incision, the Compound 1-treated cohort exhibited mechanical withdrawal thresholds (32-35 g) similar to pre-injury baseline (32 g) whereas the celecoxib, morphine and vehicle groups exhibited hyperalgesia (13-18 g) relative both to the respective cohort baseline (pre-injury) threshold values (29-31 g) and comparable to the vehicle groups post-injury (12-17 g).

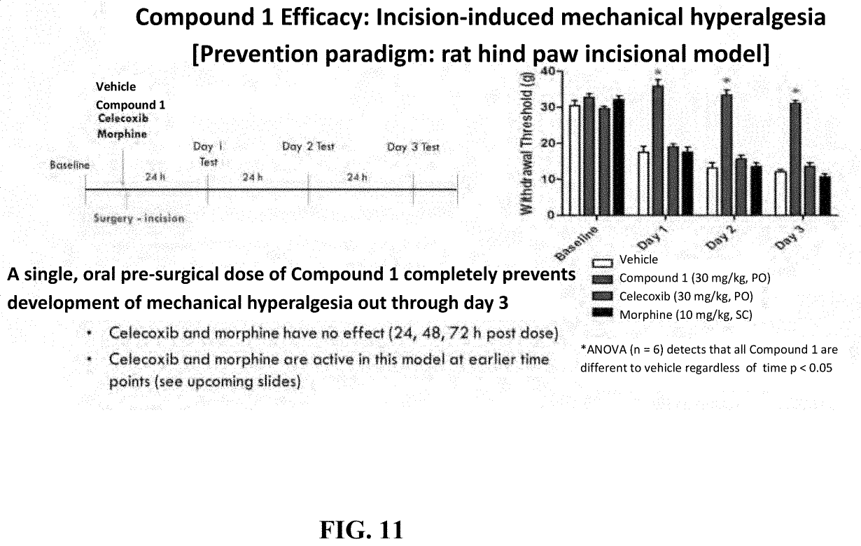

This study demonstrates that a single oral dose of Compound 1 prevents the development of incisional hyperalgesia for at least 3 days. The efficacy of Compound 1 is dose-dependent with 10 mg/kg PO producing a statistically significant effect, and 30 mg/kg PO giving full efficacy (i.e., no hyperalgesia develops), relative to vehicle (FIG. 11).

### Example 11: Reversal of Established Hyperalgesia by Compound 1

Using the Brennan protocol and a treatment paradigm in rat, Compound 1 (10 and 30 mg/kg PO), morphine (3 mg/kg SC) or vehicle were administered daily at 24 h post-incision, and again at 48 and 72 h post-incision. Mechanical thresholds were obtained at 1 h and 2 h post-dose in all cohorts on all days. Prior to incision, all cohorts exhibited normal baseline values (31-35 g) and 24 h after incision, but prior to test article administration, all cohorts exhibited a robust hyperalgesia (15-19 g; indicated as Pre-Drug (D1)).

At the Day 1 dosing, the morphine-treated cohort exhibited statistically-significant analgesia at the 1-h time point evident by threshold values (45 g) that exceed normal baseline (35 g) and this effect waned at the 2-h time point (although the animals did not exhibit hyperalgesia). The high-dose Compound 1 cohort exhibited a partial reversal of hyperalgesia at 1 h (26 g) relative to pre-drug (18 g) and compared to vehicle (18 g) and nearly a full reversal (27 g) at the 2-h time point. The low-dose Compound 1 cohort showed a partial reversal at the 2-h time point.

On Day 2 prior to dosing (Pre-Drug (D2)), the vehicle and morphine cohorts were hyperalgesic (<15 g); in the latter case, the analgesic effects of morphine from the previous dose had completely waned. In contrast, the high-dose Compound 1 cohort was essentially non-hyperalgesic (29 g) and the low-dose Compound 1 group exhibited only a mild hyperalgesia (26 g) relative to their respective baseline thresholds.

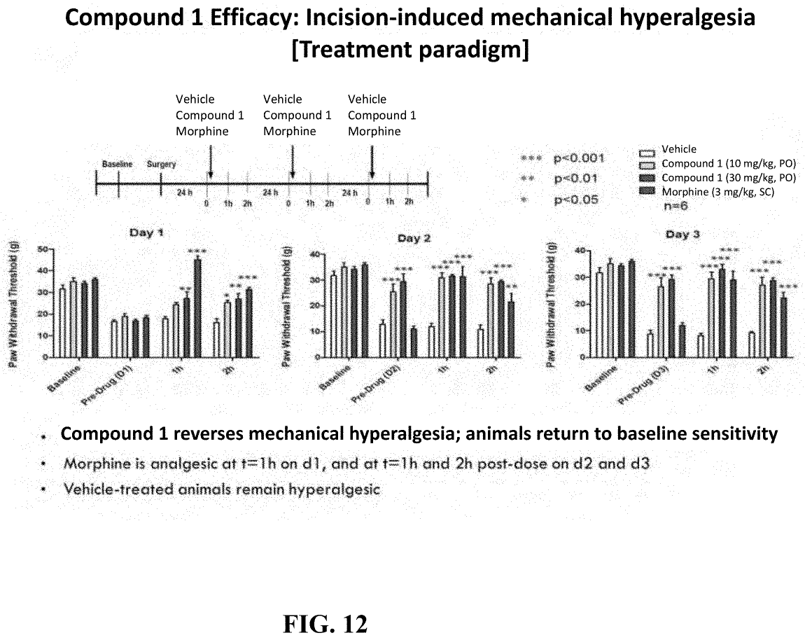

Subsequent test article dosing on Day 2 followed by mechanical threshold testing at 1 h and 2 h post-dosing demonstrated full reversal of hyperalgesia by both Compound 1 treated cohorts. The morphine-treated group was not hyperalgesic at the 1-h time point but exhibited a mild hyperalgesia at 2 h. On Day 3 prior to dosing, the Compound 1 treated animals did not show hyperalgesia, consistent with the findings from Day 2. As previously, Compound 1 (both low- and high-dose) prevented/reversed hyperalgesia completely (FIG. 12).

### Example 12: Prevention of Hyperalgesia by Compound 1

In a mouse incisional model (mimicking the Brennan protocol), animals were baselined for mechanical thresholds prior to incision and then given a daily dose of Compound 1 (10 mg/kg IP QD) or vehicle for 7 days. On Days 1 and 3 (˜24 h and ˜72 h after incision), the Compound 1 treated cohort was not hyperalgesic whereas the vehicle treated group exhibited a severe hyperalgesia. On Day 7, both cohorts exhibited normal baseline sensitivities indicating that the lesion had healed sometime after Day 3 and before Day 7, and that there were no lasting effects caused by the incision or by Compound 1 therapy.

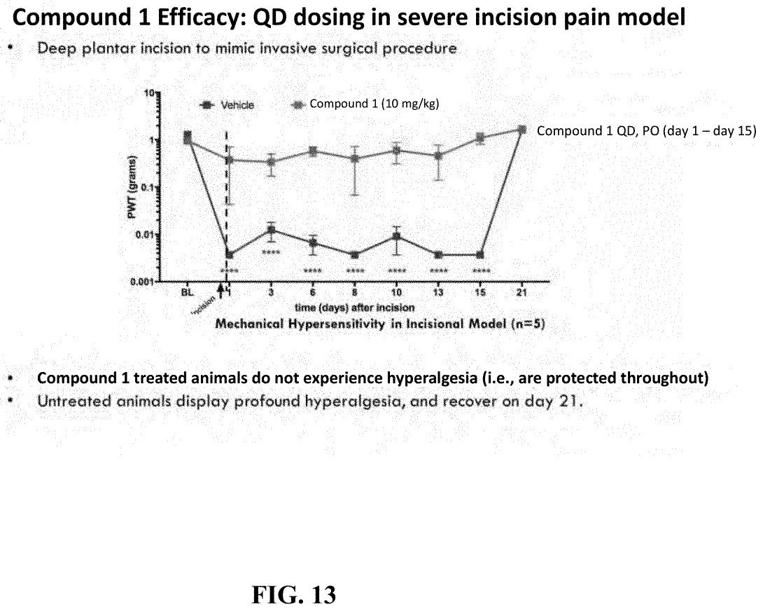

In a mouse model of severe incision (tissue and muscle spread with forceps), animals were at baseline for mechanical thresholds prior to incision and then given a daily dose of Compound 1 (10 mg/kg IP) or vehicle for 15 days. On Days 1, 3, 6, 8, 10, 13, 15 and 21 after incision, the Compound 1 treated cohort was protected from hyperalgesia whereas the vehicle-treated group exhibited a sustained hyperalgesia. On Day 21, both cohorts exhibited normal baseline sensitivities indicating that the lesion had healed sometime after Day 15 and before Day 21, and that there were no lasting effects caused by the incision or by daily oral Compound 1 therapy for 15 days (FIG. 13).

### Example 13: Compound 1 Effect on Allodynia in Rodent Models of Diabetic Neuropathy

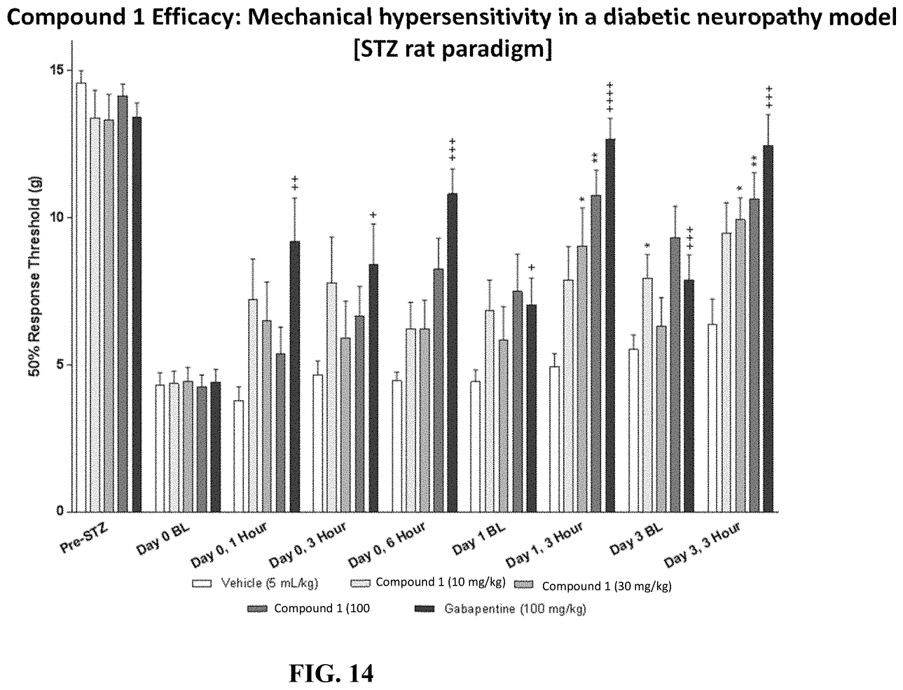

Experiments were conducted to determine the effect of Compound 1 in streptozotocin (STZ)-induced diabetic rats. In the first experiment, after obtaining mechanical threshold baselines, STZ (50 mg/kg IV) was administered (Day −7). Two days later, blood glucose levels were measured and animals that were hyperglycemic (>250 mg/dL) continued in the study. On Day 0 the baseline for mechanical thresholds prior to dosing with test articles was established for the rats. Compound 1 (10, 30, and 100 mg/kg PO), gabapentin (100 mg/kg PO) or vehicle was then administered daily for 5 days. On Day 0 mechanical thresholds were obtained at 1, 3, and 6 h post-dose. On Day 1 mechanical thresholds were measured immediately prior to dosing and then 3 h post-dose. The same regimen was followed on Day 3. The gabapentin cohort exhibited a significant reversal of allodynia 1 h post-dose on Day 0 and all time points thereafter. Compound 1-treated groups showed non-significant reversal of allodynia on Day 0 which became statistically-significant on subsequent days, evident by increases in mechanical threshold values relative to vehicle, and these effects were comparable to gabapentin (FIG. 14).

In a subsequent study, the same STZ paradigm was followed. However, in addition to the 100 mg/kg Compound 1 group, two additional cohorts received the Compound 1 100 mg/kg dose split into two or three doses per day with 50 mg/kg BID and 33 mg/kg TID. In addition to the gabapentin (100 mg/kg PO) group, a separate cohort received duloxetine (30 mg/kg PO). Additionally, in this study mechanical threshold testing was implemented every day immediately prior to dosing and 2 h post-dose for 7 days. Compound 1 reversed allodynia upon continued daily dosing reaching statistical significance from Day 1 and beyond. There was no clear difference between Compound 1-treated groups regarding dosing regimen (100 mg/kg QD vs 50 mg/kg BID vs 33 mg/kg TID). Gabapentin robustly reversed the allodynia throughout the study whereas the duloxetine cohort required successive doses for appreciable activity.

### Example 14: Methods of Analysis for Pharmacokinetics Measurements

Formulation analysis and bioanalytical methods were validated for all GLP studies. Formulation of Compound 1 in water was validated over a range of 1 to 200 mg/mL using HPLC and in 0.5% hydroxypropyl methylcellulose (HPMC) over the same range.

Compound 1 concentration in rat plasma was validated using LC/MS/MS with a lower limit of quantitation of 0.1 μg/mL using a 50 μL sample. Similar conditions were used to validate a bioanalytical method in dog plasma also using 50 μL of plasma. In both assays, a deuterated (Compound 1-d4) internal standard was used.

Dose formulation analysis for all good laboratory practice (GLP) general toxicology studies was performed using a validated high-performance liquid chromatography (HPLC)/ultraviolet (UV) analytical method (2750-001-001 Dose Formulation Method 1). The vehicle used in the in vivo toxicology studies was 0.5% HPMC). The analytical method utilized HPLC with monitoring at 227 nm with an isocratic mobile phase of methanol with the column temperature set to 25° C. Linearity over a range of 1.0 to 200 mg/mL. Dose formulations over this range were stable at room temperature for up to 13 days and were stable when stored frozen at −20° C. for up to 85 days.

In addition, formulation analysis for in vitro genetic toxicology studies was performed using a validated HPLC/UV analytical method (2750-001-001 Dose Formulation Method No. 2) with linearity over a range of 0.001 to 50 mg/mL. Dose formulations for in vitro assays were stable at room temperature for up to 1 day and when stored frozen at −20° C. for up to 45 days.

### Example 15: Rat Pharmacokinetics after Single PO Dose Administration

The objective of this non-GLP study was to determine the pharmacokinetics of Compound 1 after a single oral (gavage) dose in rats, and to determine if Compound 1 undergoes epimerization in the rat. Compound 1 was administered to CD® IGS [Crl:CD(SD)] (Sprague Dawley) rats (5/sex/group) as a single oral gavage dose of 100 mg/kg in a vehicle of 0.5% aqueous HPMC. Rats were dosed at a dose volume of 10 mL/kg body weight. Blood samples for determination of plasma levels of the test article were obtained under anesthesia from the retro-orbital sinus of each rat at six time points (30 and 60 minutes and 2, 4 and 8 and 24 hours) post-dose; EDTA was used as the anticoagulant.

Analysis using a chiral column did not provide any evidence of epimerization of Compound 1. Therefore, the samples were re-analyzed using a non-chiral method for improved quantitation. The test article was quantifiable at all time points through 24 hours for all five females. For males, the test article was quantifiable at all time points for all five animals through 8 hours, but at the 24 hours post-dose time point results were quantifiable for only two of the five animals. The mean Cₘₐₓ values were 60 and 15 μg/mL for females and males, respectively. Tₘₐₓ was 2 hours for females and 1 hour for males, with t_{1/2} values of 4 and 7 hours, respectively.

TABLE 13
Compound 1 Concentration in Rat Plasma (Single Dose PO Study)

| | | Blood Collection Time Point (Hours Post-Dose) | | | | | |
|---|---|---|---|---|---|---|---|
| | | 0.5 | 1 | 2 | 4 | 8 | 24 |
| Group | Animal ID | Compound 1 Concentration (μg/mL) | | | | | |
| Male | 1 | 14.0 | 14.8 | 14.0 | 1.56 | 1.57 | BQL |
| 100 mg/kg | 2 | 11.4 | 11.0 | 14.9 | 6.65 | 1.53 | 1.04 |
| | 3 | 10.8 | 11.8 | 10.7 | 3.81 | 3.05 | BQL |
| | 4 | 13.8 | 14.3 | 16.3 | 4.82 | 1.61 | 0.157 |
| | 5 | 17.5 | 22.6 | 15.7 | 13.9 | 2.81 | BQL |
| | Average: | 13.5 | 14.9 | 14.3 | 6.15 | 2.11 | 0.599 |
| | STD: | 2.6 | 4.6 | 2.2 | 4.7 | 0.75 | 0.62 |
| | % RSD: | 20 | 31 | 15 | 77 | 35 | 104 |
| Female | 6 | 34.3 | 53.9 | 58.2 | 14.4 | 1.78 | 0.250 |
| 100 mg/kg | 7 | 36.4 | 50.1 | 63.1 | 14.4 | 1.80 | 0.240 |
| | 8 | 44.7 | 63.6 | 58.9 | 31.5 | 4.14 | 0.489 |
| | 9 | 43.2 | 63.2 | 60.1 | 32.4 | 4.21 | 0.482 |
| | 10 | 45.4 | 69.7 | 72.8 | 20.4 | 2.23 | 0.428 |
| | Average: | 40.8 | 60.1 | 62.6 | 22.6 | 2.83 | 0.378 |

Cₘₐₓ and AUC values did not differ when these same samples were analyzed using a non-chiral assay. The effect of food on oral bioavailability was determined. Compound 1 was well and rapidly absorbed in both fed and fasted rats at both 10 and 100 mg/kg doses. No statistically significant differences in pharmacokinetic properties were found between the fed and fasted rats in the 100 mg/kg oral dosing groups. However, significantly increased peak plasma concentrations were observed in the fasted rats compared to the fed rats at the 10 mg/kg dose. A secondary peak in the concentration-time profiles was detected suggesting enterohepatic circulation, probably via formation of glucuronide conjugates of Compound 1.

### Example 16: Rat Pharmacokinetics after 7-Day Repeat Dose PO Administration

For the first 7-day repeat dose study, Hsd:Sprague Dawley®™SD®™ Rats (SD) (4/sex/group) were administered Compound 1 by oral gavage for 7 days at dose levels of 10, 100 and 1000 mg/kg body weight. The dose volume was 10 mL/kg for the control and 100 mg/kg/day groups and 20 mL/kg for the 1000 mg/kg/day group. The vehicle was 0.5% HPMC. Blood was collected for toxicokinetic analysis 2 hours post-dose on the first and last day of dosing. Gross necropsy evaluation, organ weights and histopathology were conducted for the 1000 mg/kg/day animals only. Blood samples for determination of plasma levels of the test article were obtained under anesthesia from the retro-orbital sinus of each rat at six time points (pre-dose; 1, 2, 4, 8 and 24 hours post-dose) on Days 1 and 7.

Based on the results of the previous investigation with Compound 1, plasma samples were analyzed using a non-chiral method for optimum quantitation. Toxicokinetic (TK) results were as follows:

TABLE 14
Compound 1 Concentration in Rat Plasma
(7 Day PO Repeat Dose Study)

| | Compound 1 | Mean TK Parameters | | | |
|---|---|---|---|---|---|
| | Dose Level | t_{1/2} | Tₘₐₓ | Cₘₐₓ | AUC₀₋₂₄ |
| Group | (mg/kg) | (hr) | (hr) | (μg/mL) | (hr*μg/mL) |

First Dose

| | | | | | |
|---|---|---|---|---|---|
| 1 | 2000 | 6.7 | 1 | 121 | 1609 |

Seventh Daily Dose

| | | | | | |
|---|---|---|---|---|---|
| 1 | 2000 | 3.9 | 2 | 119 | 1367 |

In summary, oral (gavage) administration of Compound 1 to female rats for 7 days at a dose level of 2000 mg/kg was tolerated. Therefore, 2000 mg/kg was considered the MTD and was recommended as the high dose level for the 28-day oral toxicity study of Compound 1 in rats since the human expected systemic exposure is unknown.

### Example 17: Rat Pharmacokinetics after 28-Day Repeat Dose PO Administration

A 28-day GLP repeat dose study with Compound 1 in rats was conducted with oral gavage doses of 0, 500, 1000 or 2000 mg/kg/day at a dosing volume of 10 mL/kg. The vehicle was 0.5% HPMC. There were 15 rats/sex in the control and high dose groups (5/sex for a 14-day recovery period) while the low and mid doses consisted of 10 rats/sex. Satellite groups of rats/sex/group for toxicokinetics were included.

Blood samples (approximately 0.5 mL) for determination of plasma levels of Compound 1 were collected from the retro-orbital plexus from TK study animals at six time points (at approximately 0.5, 1, 2, 4, 8 and 24 hours post-dose) on Days 1, 14 and 28. Rats in Group 1 (Vehicle Control) were bled once at approximately 1 hour post-dose on those days.

Toxicokinetic analysis revealed that after oral administration of a single dose of Compound 1 ranging from 500 to 2000 mg/kg, median tₘₐₓ on Days 1, 14 and 28 was 1 hour (0.5 to 2 hours), with no differences between sexes or dose levels. Median tₘₐₓ varied somewhat with study day, however, across all dose levels and sexes, overall median tₘₐₓ was 2 hours on Day 1, and 1 hour on Days 14 and 28. Overall mean t_{1/2} was 4.24 hours, with little or no differences between sexes, study days or dose levels. Overall mean CL/F was 2.11 L/hr/kg (moderate degree of variability) and was somewhat lower in females compared to males.

There were no consistent differences in CL/F between study days or dose levels. Overall mean V/F was 12.5 L/kg (high degree of variability) and was lower in females than males. There were no consistent differences in V/F between study days or dose levels. All measures of systemic exposure (Cₘₐₓ, AUC_{TAU} and AUC_{INF}) were higher in females than in males at all doses on Days 1, 14 and 28. Compound 1 concentrations did not accumulate from Day 1 to Day 14 in either sex at any dose level, but instead appeared to decrease slightly. However, Compound 1 concentrations did accumulate from Day 14 to Day 28, particularly in females. Over the entire 28-day period, there was no net change in systemic exposure. Systemic exposure increased with dose in both sexes on all TK analysis days, although this increase was not always proportional to dose.

TABLE 15
Selected Toxicokinetic Parameters from a 28-Day Rat Study (PO)

| | | Dose | tₘₐₓ | Cₘₐₓ | AUC_{TAU} | AUC_{INF} | t_{1/2} | CL/F¹ | V/F² |
|---|---|---|---|---|---|---|---|---|---|
| Day | Sex | (mg/kg) | (hr) | μg/mL | (hr* μg/mL) | (hr* μg/mL) | (hr) | (L/hr/kg) | (L/kg) |
| 1 | F | 500 | 2 | 74.8 | 298 | 299 | 3.36 | 1.67 | 8.10 |
| | | 1000 | 2 | 108 | 788 | 794 | 3.39 | 1.26 | 6.15 |
| | | 2000 | 2 | 118 | 1161 | 1324 | 8.05 | 1.51 | 17.6 |
| | M | 500 | 1 | 55.1 | 270 | 275 | 4.18 | 1.82 | 11.0 |
| | | 1000 | 1 | 52.7 | 456 | 460 | 3.46 | 2.17 | 10.9 |
| | | 2000 | 2 | 83.1 | 768 | 771 | 2.81 | 2.59 | 10.5 |
| 14 | F | 500 | 1 | 75.0 | 223 | 225 | 4.20 | 2.25 | 13.6 |
| | | 1000 | 2 | 96.3 | 430 | 437 | 4.19 | 2.32 | 14.0 |
| | | 2000 | 1 | 127 | 942 | 952 | 3.56 | 2.12 | 10.9 |
| | M | 500 | 1 | 45.8 | 244 | 258 | 6.27 | 2.05 | 18.5 |
| | | 1000 | 1 | 51.9 | 293 | 296 | 3.68 | 2.32 | 18.1 |
| | | 2000 | 1 | 75.6 | 701 | 709 | 3.66 | 2.12 | 15.1 |
| 28 | F | 500 | 0.5 | 86.4 | 297 | 303 | 4.62 | 1.68 | 11.2 |
| | | 1000 | 1 | 128 | 736 | 741 | 3.20 | 1.36 | 6.26 |
| | | 2000 | 0.5 | 139 | 1305 | 1327 | 3.93 | 1.53 | 8.70 |
| | M | 500 | 1 | 48.6 | 305 | 333 | 6.90 | 1.64 | 16.4 |
| | | 1000 | 2 | 47.9 | 334 | 337 | 3.31 | 2.99 | 14.3 |
| | | 2000 | 2 | 83.6 | 724 | 731 | 3.53 | 2.76 | 14.1 |

¹CL/F = CLz/F on Day 1 and Clss/F on Days 14 and 28
²V/F = Vz/F on Day 1 and Vss/F on Days 14 and 28

### Example 18: 7 Day Repeat IV Dose Study in the Rat

In a third study, SD rats (3/sex/group) were administered Compound 1 IV via the tail vein for 7 days at doses of 0, 75 or 150 mg/kg/day. The vehicle was 15% dimethylacetamide, 85% phosphate buffered saline. Blood was collected from all surviving animals for toxicokinetic analysis approximately 2 hours after the last dose on Day 7.

TABLE 16
Compound 1 Concentration in Rat Plasma
in a 7 Day Repeat Dose IV Study

| | | | Group | | | Mean | |
|---|---|---|---|---|---|---|---|
| Animal | | Gen- | Time | Conc | Conc | Conc | Conc |
| Dose | Group | der | (h)* | (ng/mL) | (μg/mL) | (μg/mL) | (μM) |
| B82944 | Control | 1 M | 2 h | BLOQ | BLOQ | BLOQ | 0.00 |
| B82945 | Control | 1 M | 2 h | BLOQ | BLOQ | | 0.00 |
| B82946 | Control | 1 M | 2 h | BLOQ | BLOQ | | 0.00 |
| B82947 | 75 mg/kg | 2 M | 2 h | 4310 | 4.31 | 3.01 | 16.18 |
| B82948 | 75 mg/kg | 2 M | 2 h | 2350 | 2.35 | | 8.82 |
| B82949 | 75 mg/kg | 2 M | 2 h | 2380 | 2.38 | | 8.94 |
| B82950 | 150 mg/kg | 3 M | 2 h | 4280 | 4.28 | 5.92 | 16.07 |
| B82951 | 150 mg/kg | 3 M | 2 h | 6910 | 6.91 | | 25.95 |
| B82952 | 150 mg/kg | 3 M | 2 h | 6560 | 6.56 | | 24.63 |
| B82953 | Control | 1 F | 2 h | BLOQ | BLOQ | BLOQ | 0.00 |
| B82954 | Control | 1 F | 2 h | BLOQ | BLOQ | | 0.00 |
| B82955 | Control | 1 F | 2 h | BLOQ | BLOQ | | 0.00 |
| B82956 | 75 mg/kg | 2 F | 2 h | 2670 | 2.67 | 3.03 | 10.03 |
| B82957 | 75 mg/kg | 2 F | 2 h | 3250 | 3.25 | | 12.20 |
| B82958 | 75 mg/kg | 2 F | 2 h | 3180 | 3.18 | | 11.94 |
| B82959 | 150 mg/kg | 3 F | 2 h | 5990 | 5.99 | 4.65 | 22.49 |
| B82960 | 150 mg/kg | 3 F | 2 h | 2430 | 2.43 | | 9.12 |
| B82961 | 150 mg/kg | 3 F | 2 h | 5530 | 5.53 | | 20.76 |

### Example 19: Dog Pharmacokinetics after Single PO and IV Dose Administration

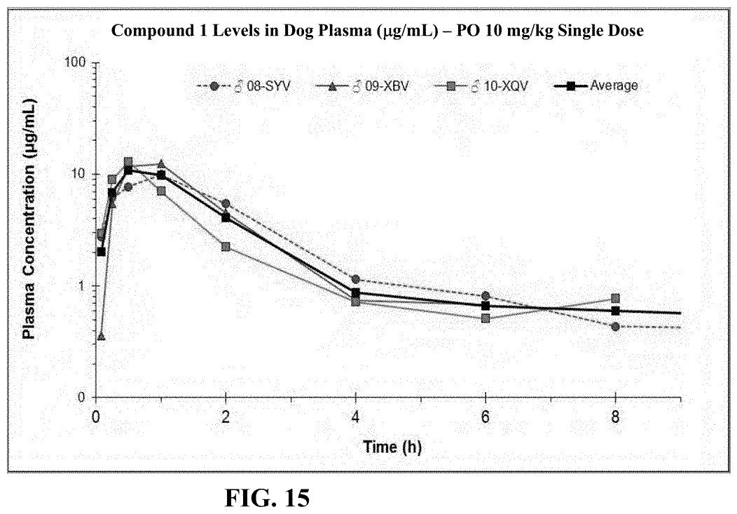

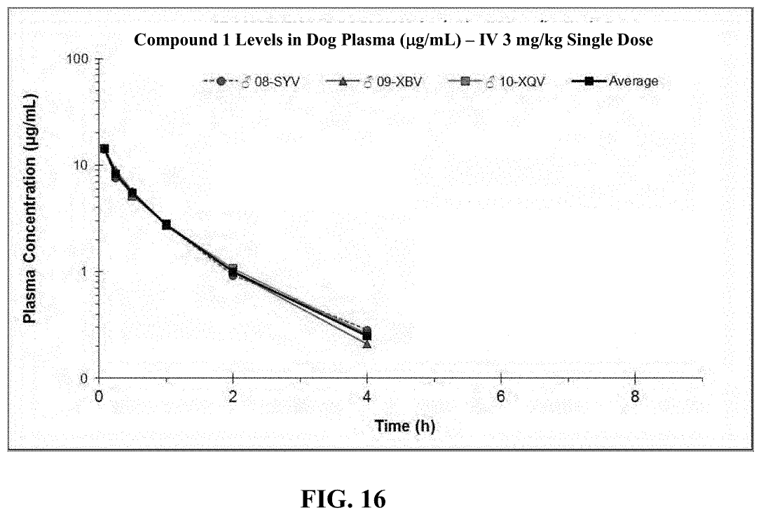

The objective of the first single dose non-GLP study in dogs was to determine the pharmacokinetics and bioavailability of Compound 1 after a single oral (gavage) or single intravenous (IV) dose in dogs. The test article formulation was administered to three male dogs orally via gavage at a dose level of 10 mg/kg (dosing volume of 2 mL/kg body weight). Following a wash-out period of a minimum of three days, the same three dogs were administered the test article formulation via an IV bolus push over approximately 1-2 minutes at a dose level of 3 mg/kg (dosing volume of 0.5 mL/kg). The vehicle for oral gavage dosing was 0.5% HPMC; the vehicle for IV injection was sterile phosphate buffered saline (PBS; pH 7.4). Blood samples for determination of plasma levels of the test article were obtained from the jugular vein at nine time points (5, 15, 30 and 60 minutes and 2, 4, 6, 8 and 24 hours) after each dose. Animals were returned to quarantine after the last blood collection. The plasma concentration over an 8 hour period for both PO and IV dose administrations is shown in FIG. 15 and FIG. 16.

Analysis using a chiral column did not provide any evidence of epimerization of Compound 1. Therefore, the samples were re-analyzed using a non-chiral method for improved quantitation. After oral administration at 10 mg/kg, the test article was quantifiable up to the 6-hour time point for all animals, but at the 24 hours post-dose time point results were quantifiable for only one animal. Average oral Cₘₐₓ was 12 μg/mL with a tₘₐₓ of about 1 hour. Median t_{1/2} was 1.4 hours with 72% mean oral bioavailability with a range of 56 to 91%. After IV administration at 3 mg/kg, the test article was quantifiable up to the 4-hour time point for all animals and tₘₐₓ was about 5 minutes. Since test article was not quantifiable at the 6-hour and subsequent time points for IV administration, Cₘₐₓ and bioavailability could not be calculated.

In another study Compound 1 was administered IV as a single dose to fasted beagle dogs at 10 mg/kg and orally at 10 and 100 mg/kg. Compound 1 was rapidly and well absorbed orally, within 1 hr, reaching peak plasma concentrations of 15 and 108 μg/mL at 10 and 100 mg/kg doses, respectively. The compound distributed readily in tissues with a moderate steady-state volume of distribution (0.3 L/kg). The compound was eliminated from the systemic circulation with a mean terminal half-life of 3.9 h. The Compound 1 had good oral bioavailability (75%) at both the 10 and 100 mg/kg doses.

The objective of a third single dose study in dogs was to determine the maximum tolerated dose (MTD) of Compound 1 following a single oral (gavage) dose in male and female dogs. Using a pyramiding dose schedule, dose levels of 250, 350 and 500 mg/kg were administered to 2 dogs/sex (after a washout period of at least three days, the same animals were dosed with the next dose level). The dose volume was 10 mL/kg body weight. The vehicle was 0.5% aqueous HPMC. Blood samples for determination of plasma levels of Compound 1 were obtained from the jugular vein of each dog at three time points (0.5, 1 and 2 hours) after each dose.

The single dose of 500 mg/kg Compound 1 was not tolerated due to emesis in three of the four dosed animals (1 male, both females). Therefore, a single oral (gavage) dose of 500 mg/kg Compound 1 in dogs was not tolerated and 400 mg/kg/was chosen as the high dose for the subsequent 7 day study in dogs.

TABLE 17
Compound 1 Concentration in Dog Plasma (Single Dose PO Study)

| | Average Compound 1 Concentration | | | | | |
|---|---|---|---|---|---|---|
| | (μg/mL) per Blood Collection Time | | | | | |
| | Point (Hour); N = 2 | | | | | |
| Compound 1 | 0.5 | | 1 | | 2 | |
| Dose Level (mg/kg) | M | F | M | F | M | F |
| 250 (Dose 1; Day 1) | 71 | 28 | 110 | 54 | 91 | 46 |
| 500 (Dose 2; Day 4) | 111 | 87 | 191 | 136 | 193 | 124 |
| 350 (Dose 3; Day 9) | 134 | 67 | 220 | 154 | 153 | 119 |

### Example 20: Dog Pharmacokinetics after 7-Day Repeat Dose PO Administration

The objective was to develop a preliminary toxicity profile of Compound 1 following daily oral (gavage) administration for 7 days to male and female dogs to support dose selection for a 28-day toxicity study in dogs. Doses were selected based on the results of the MTD phase. Dose levels were 0 (Vehicle Control), 200, 350 and 400 mg/kg (1 dog/sex/group). The vehicle was 0.5% aqueous HPMC.

Blood samples for determination of plasma levels of Compound 1 were obtained from the jugular or cephalic vein at seven time points [0 (pre-dose); 0.5, 1, 2, 4, 8 and 24 hours post-dose] on Day 1 and at six time points (same as for Day 1 but excluding pre-dose) on Day 7. The animals used in this study were returned to the holding colony

TABLE 18
Compound 1 Concentration in Dog Plasma
(7 Day Repeat Dose PO Study)

| | TK Parameter; N = 1 | | | | | | | |
|---|---|---|---|---|---|---|---|---|
| Compound 1 | t_{1/2} | | Tₘₐₓ | | Cₘₐₓ | | AUC₀₋ₗₐₛₜ | |
| Dose Level | (hr) | | (hr) | | (μg/mL) | | (hr*μg/mL) | |
| (mg/kg) | M | F | M | F | M | F | M | F |

Day 1

| | | | | | | | | |
|---|---|---|---|---|---|---|---|---|
| 200 | 1.1 | 1.3 | 2.0 | 2.0 | 150 | 152 | 538 | 582 |
| 350 | 1.1 | 1.2 | 2.0 | 2.0 | 176 | 228 | 613 | 834 |
| 400 | 1.2 | 3.9 | 2.0 | 1.0 | 167 | 109 | 657 | 464 |

Day 7

| | | | | | | | | |
|---|---|---|---|---|---|---|---|---|
| 200 | 1.4 | 1.5 | 1.0 | 2.0 | 121 | 103 | 540 | 411 |
| 350 | 1.2 | 2.4 | 1.0 | 0.5 | 184 | 207 | 591 | 738 |
| 400 | 1.3 | 2.5 | 1.0 | 2.0 | 69 | 183 | 210 | 751 |

### Example 21: Dog Pharmacokinetics after 28-Day Repeat Dose PO Administration

Beagle dogs (4/sex/group plus 2/sex/group for control and high dose animals for recovery) were administered daily doses of 0, 100, 250 or 400 mg/kg/day of Compound 1 in 0.5% HPMC. Blood samples (approximately 3 mL) for determination of plasma levels of Compound 1 were collected from the jugular or cephalic vein of the Main Study animals at seven time points (pre-dose, and at approximately 0.5, 1, 2, 4, 8 and 24 hours post-dose) relative to dosing on Days 1, 14 and 28. Dogs in Group 1 (Vehicle Control) were bled once at approximately 2-4 hours post-dose on those days.

Compound 1 absorption was similar across all animals in the study. Median tₘₐₓ was 1 hour, with no obvious relation to study day or sex. Tₘₐₓ varied slightly by dose level; median tₘₐₓ was 1 hour for the 100 and 250 mg/kg treatment groups but increased to 2 hours for the 400 mg/kg treatment group. Across both sexes, study days and dose levels, mean t_{1/2} was 3.5 hours. There was little difference in mean t_{1/2} between females and males, or among study days. Mean t_{1/2} tended to decrease as dose increased.

CL/F and V/F tended to increase as dose increased. Across all study days and dose levels, there was very little difference in systemic exposure between females and males. Not only was there no accumulation, there also was very little overall change in plasma levels and systemic exposure from Day 1 to Day 15 to Day 28, regardless of sex or dose level. Compound 1 systemic exposure increased with dose in a manner that was less than proportional to dose.

TABLE 19
Compound 1 Concentration in Dog Plasma
(28 Day Repeat Dose PO Study

| | Dose | Sex | Cₘₐₓ | AUC_{TAE} | AUC_{INF} |
|---|---|---|---|---|---|
| Day | (mg/kg) | ratio | (μg/mL) | (hr*μg · mL) | (hr*μg · mL) |
| 1 | 100 | F | 69.3 | 198 | 200 |
| | | M | 49.9 | 171 | 172 |
| | | F/M | 1.39 | 1.16 | 1.16 |
| | 250 | F | 101 | 423 | 439 |
| | | M | 104 | 393 | 396 |
| | | F/M | 0.97 | 1.07 | 1.31 |
| | 400 | F | 129 | 512 | 514 |
| | | M | 128 | 618 | 622 |
| | | F/M | 1.00 | 0.83 | 0.83 |
| 15 | 100 | F | 53.0 | 205 | 207 |
| | | M | 50.4 | 166 | 168 |
| | | F/M | 1.05 | 1.23 | 1.23 |
| | 250 | F | 94.6 | 364 | 366 |
| | | M | 88.0 | 361 | 364 |
| | | F/M | 1.08 | 1.01 | 1.01 |
| | 400 | F | 123 | 545 | 546 |
| | | M | 112 | 456 | 458 |
| | | F/M | 1.10 | 1.20 | 1.19 |
| 28 | 100 | F | 60.8 | 200 | 201 |
| | | M | 45.0 | 177 | 180 |
| | | F/M | 1.35 | 1.13 | 1.12 |
| | 250 | F | 89.5 | 360 | 362 |
| | | M | 105 | 407 | 409 |
| | | F/M | 0.85 | 0.88 | 0.88 |
| | 400 | F | 131 | 627 | 632 |
| | | M | 125 | 470 | 472 |
| | | F/M | 1.05 | 1.34 | 1.34 |

### Example 22: Distribution of Compound 1

A rat brain distribution study performed in order to determine standard pharmacokinetic parameters and to assess the brain penetration properties of Compound 1 administered orally and intravenously. The compound was rapidly and completely absorbed orally in rats after administration of a 30 mg/kg dose, reaching a peak plasma concentration of 9 μg/mL within 1 hr. The compound distributed readily in tissues with a steady-state volume of distribution of 1.7 L/kg. Compound 1 was peripherally restricted with a brain-to-plasma concentration ratio of 0.02 1 h after IV administration eliminated at a moderate rate from the systemic circulation. Oral bioavailability of 111% was calculated for the 30 mg/kg oral dose with a terminal half-life of 1.6 hours.

A Red Blood Cell (RBC) partitioning showed that Compound 1 poorly partitions into RBC at 60 min post-exposure (K_{RBC/PL}<0.25) across all species (rat, dog, monkey, human).

### Example 23: Metabolism of Compound 1

In hepatocytes from rat, dog, monkey and humans, Compound 1 showed metabolic stability and none of the predicted metabolites appeared to have the potential to be reactive. Compound was also stable in plasma from a variety of species. Examination of urine and plasma in a rat single dose study demonstrated that approximately 5-15% of administered Compound 1 was recovered unchanged in both urine and feces. A small amount (1-2%) of the compound was found as the glucuronide while the remainder (approximately 85%) was a sulfate conjugate of the parent, excreted in both the urine and feces. Further metabolism work in additional species will be undertaken.

### Example 24: CYP Inhibition

At varying concentrations, Compound 1 was incubated with human liver microsomes (HLM) in the presence of known substrates (see below) of specific CYP isoforms, in order to measure inhibition induced by Compound 1. Microsomes were incubated with known inhibitors (positive controls) of each CYP isoform, in the presence of substrate, in order to measure the metabolic activity of the microsomes.

TABLE 20
CYP Inhibition

| Test compound | Compound 1 |
|---|---|
| Inhibitor | CYP1A2: α-Naphthoflavone |
| (Positive control) | CYP2C9: Sulfaphenazole |
| | CYP2C19: Omeprazole |
| | CYP2D6: Quinidine |
| | CYP3A4: Ketoconazole |
| Final highest | 100 μM test compound, 20 μM α-Naphthoflavone; |
| concentration | 10 μM Sulfaphenazole;100 μM Omeprazole; 2.5 μM |
| | Quinidine; 2.5 μM Ketoconazole |
| Substrate | CYP1A2: Phenacetin |
| | C YP2C9: Tolbutamide |
| | CYP2C19: S-Mephenytoin |
| | CYP2D6: Dextromethorphan |
| | CYP3A4: Midazolam |
| Test concentration | 30 μM Phenacetin; 100 μM Tolbutamide; 35 μM |
| | S-Mephenytoin; 5 μM Dextromethorphan; 5 μM |
| | Midazolam |
| Test systems | Human liver microsomes from BD Gentest (0.5 |
| | mg/mL for CYP1A2, 2C9, 2C19; 0.2 mg/mL for |
| | 2D6; 0.1 mg/mL for CYP3A4) |
| Incubation condition | 37° C. incubation for 10 minutes for CYP1A2; |
| | 15 minutes for CYP2C9, 2D6; 45 minutes for |
| | CYP2C19; 5 minutes for CYP3A4 |
| Sample size | Duplicate (n = 2) |
| Bioanalytical | HPLC-MS/MS |
| method | |

Compound 1 did not inhibit the five CYP isoforms tested (see table below). The positive controls produced CYP inhibition consistent with historical (and literature) values indicating that the microsomes were metabolically active and of high integrity.

| CYP isoform | IC₅₀ (μM) |
|---|---|
| CYP 3A4 | >100 |
| CYP 1A2 | >100 |
| CYP 2D6 | >100 |
| CYP 2C9 | >100 |
| CYP 2C19 | >100 |

### Example 25: Inhibition of Transporters

Compound 1 was evaluated to determine inhibition of human ATP binding cassette (ABC) transporters (known as efflux transporters) and solute-linked carrier (SLC) transporters (known as uptake transporters as outlined below:

TABLE 21
Inhibition of Transporters

| Transporter | | | |
|---|---|---|---|
| (Gene | Test system | Probe substrate | Experimental design |
| P-gp | Caco-2 cells | Digoxin | Bidirectional transport of the probe substrate |
| (MDR1/ABCB1 | | | across Caco-2 cells, MDCKII-BCRP cells |
| | | | and MDCKII control cells |
| BCRP | MDCKII cells | Prazosin | |
| (ABCG2) | | | |
| OATP1B1 | HEK293 cells | [³H]-Estradiol-17β- | Accumulation of the probe substrate into |
| (OATP2/OATP - | | glucuroni de | transporter-expressing and control cells |
| C/SLCO1B1) | | | |
| OATP1B3 | | | |
| (OATP8/SLCO1 | | | |
| B3) | | | |
| OAT1 | | [³H]-p-Aminohippurate | |
| (SLC22A6) | | | |
| OAT3 | | [³H]-Estrone-3-sulfate | |
| (SLC22A8) | | | |
| OCT1 | | [¹⁴C]- | |
| (SLC22A1) | | Tetraethylammonium | |
| MATE1 | | [¹⁴C]-Metformin | |
| (SLC47A1) | | | |
| MATE2-K | | | |
| (SLC47A2) | | | |

TABLE 22
Experimental Design for the In Vitro Evaluation
of Compound 1 for Inhibition of P-gp and BCRP

| | Caco-2 | MDCKII-BCRP |
|---|---|---|
| Test article | Compound 1 | Compound 1 |
| [Test article] (μM) | 1, 3, 10, 30, 100, 600 | 0.1, 0.3, 1, 3, 10, 30 |
| Probe substrate | Digoxin (10 μM) | Prazosin (1 μM) |
| Positive control | Valspodar (1 μM) | Ko143 (1 μM) |
| inhibitor(s) | | |
| | Verapamil (60 μM) | Lopinavir (30 μM) |
| Permeability control | Lucifer yellow | Not applicable |
| | (40 μg/mL) | |
| Nominal cell | 0.3 × 10⁵ | 0.3 to 0.4 × 10⁶ |
| number per well | | |
| Volume per well | Apical: 200; | Apical: 200; |
| (μL) | Basal: 980 | Basal: 980 |
| Preincubation time | 30 to 60 | 30 to 60 |
| (min) | | |
| Incubation time | Donor: 0, 120; | Donor: 0, 120; |
| (min) | Receiver: 120 | Receiver: 120 |
| Incubation tem- | 37 ± 2 | 37 ± 2 |
| perature (° C.) | | |
| Incubation medium | HBSS supplemented | HBSS supplemented |
| | with HEPES (25 mM) | with HEPES (25 mM) |
| | and glucose (25 mM) | and glucose (25 mM) |
| Number of replicates | 3 | 3 |
| Probe substrate | LC-MS/MS | LC-MS/MS |
| analysis method | | |

TABLE 23
Experimental Design for the In Vitro Evaluation of Compound
1 for the Inhibition of OATP, OAT, OCT and MATE inhibition

| | | | | | MATE1/ |
|---|---|---|---|---|---|
| | OATP1B1/ | OAT1 | OAT3 | OCT1 | MATE2-K |
| Test article | Compound 1 | Compound 1 | Compound 1 | Compound 1 | Compound 1 |
| [Test article] | 0.3, 1, 3, 10, 30, | 0.3, 1, 3, 10, 30, | 0.3, 1, 3, 10, 30, | 0.3, 1, 3, 10, 30, | 0.3, 1, 3, 10, 30, |
| (μM) | 100, 600 | 100, 600 | 100, 600 | 100, 600 | 100, 600 |
| Probe | [³H]-Estradiol- | [³H]-p- | [³H]-Estrone-3- | [¹⁴C]-Tetraethyl- | [¹⁴C]-Metformin |
| substrate | 17β-glucuronide | Aminohippurate | sulfate | ammonium bromide | (10 μM) |
| | (0.05 μM) | (1 μM) | (0.05 μM) | (5 μM) | |
| Positive | Rifampin | Probenecid | Probenecid | Quinidine | Pyrimethamine |
| control | (10 μM) | (100 μM) | (100 μM) | (100 μM) | (0.1 μM for |
| inhibitor(s) | | | | | MATE1 and |
| | | | | | 0.3 μM for |
| | | | | | MATE2-K) |
| | Cyclosporine | Novobiocin | Ibuprofen | Verapamil | Cimetidine |
| | (1 μM) | (300 μM) | (100 μM) | (10 μM) | (10 μM for |
| | | | | | MATE1 and |
| | | | | | 300 μM for |
| | | | | | MATE2-K)) |
| Nominal cell | 0 2. to 04 × 10⁶ | 0 2 to 0 4 × 10⁶ | 0 2 to 0 4 × 10⁶ | 0 2 to 0 4 × 10⁶ | 0 2 to 0 4 × 10⁶ |
| number per | | | | | |
| well | | | | | |
| Volume per | 300 | 300 | 300 | 300 | 300 |
| well (μL) | | | | | |
| Pre-incubation | 30 | 15 | 15 | 15 | 15 |
| time (min) | | | | | |
| Incubation | 2 | 1 | 2 | 15 | 5 |
| time (min) | | | | | |
| Incubation | 37 ± 2 | 37 ± 2 | 37 ± 2 | 37 ± 2 | 37 ± 2 |
| temperature | | | | | |
| (° C.) | | | | | |
| Incubation | HBSS | HBSS | HBSS | HBSS | HBSS |
| medium | supplemented | supplemented | supplemented | supplemented | supplemented |
| | with HEPES | with HEPES | with HEPES | with HEPES | with HEPES |
| | (9 mM) and | (9 mM) and | (9 mM) and | (9 mM) and | (9 mM) and |
| | sodium | sodium | sodium | sodium | sodium |
| | bicarbonate | bicarbonate | bicarbonate | bicarbonate | bicarbonate |
| | (4 mM), pH 7.4 | (4 mM), pH 7.4 | (4 mM), pH 7.4 | (4 mM), pH 7.4 | (4 mM), pH 8.5 |
| Number of | 3 | 3 | 3 | 3 | 3 |
| replicates | | | | | |
| Probe | LSC | LSC | LSC | LSC | LSC |
| substrate | | | | | |
| analysis | | | | | |
| method | | | | | |

The toxicity of Compound 1 to the various cell systems used in the study were assessed by measuring the lactate dehydrogenase (LDH) released from the cells into the medium. For Caco-2 and HEK293 cells, less than 25% cytotoxicity was observed. In MDCKII control cells, 100 and 600 μM Compound 1 were cytotoxic with percent cytotoxicity of 31.3 and 33.6%, respectively. As a result, 30 μM Compound 1 was the highest concentration analyzed for BCRP inhibition.

| Transporter | IC₅₀ (μM) |
|---|---|
| P-gp | >600 |
| BCRP | >30 |
| OATP1B1 | >600 |
| OATP1B3 | >600 |
| OAT1 | >600 |
| OAT3 | 174 |
| OCT1 | >600 |
| MATE1 | >600 |
| MATE2-K | >600 |

### Example 26: Excretion

After intravenous administration, Compound 1 was eliminated from the plasma with a half-life of approximately 3 hours in rats. After oral administration, the t_{1/2} ranged from 3-8 hours in rats and approximately 3.5 hours in dogs. In a study in rats Compound 1 was excreted in both urine and feces primarily as a sulfate conjugate with a small amount excreted unchanged.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Enumerated Embodiments

The following enumerated embodiments are provided, the numbering of which is not to be construed as designating levels of importance:

Embodiment 1 provides a method of treating diabetic neuropathy, the method comprising administering a therapeutically effective amount of a composition comprising a compound of Formula I:

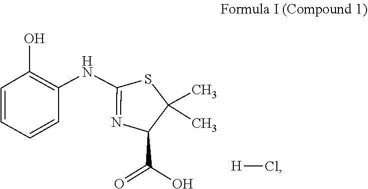 [SMILES: CC1(C)SC(Nc2ccccc2O)=N[C@@H]1C(=O)O ; Cl]

to an individual having diabetic neuropathy.

Embodiment 2 provides the method of embodiment 1, wherein the diabetic neuropathy comprises peripheral neuropathy, proximal neuropathy, autonomic neuropathy, focal neuropathy, or combinations thereof.

Embodiment 3 provides the method of any one of embodiments 1-2, wherein the individual has type I or type II diabetes.

Embodiment 4 provides the method of any one of embodiments 1-3, wherein the therapeutically effective amount of Compound 1 comprises about 5 mg to about 5000 mg.

Embodiment 5 provides the method of any one of embodiments 1-4, wherein composition is administered for about 1 day to about 90 days.

Embodiment 6 provides the method of any one of embodiments 1-5, wherein administration of the composition results in a maximum observed plasma concentration (Cₘₐₓ) of about 5 μg/mL to about 300 μg/mL.

Embodiment 7 provides the method of any one of embodiments 1-6, wherein administration of the composition results in an area under the curve (AUC_{INF}) of about 100 hr·μg/mL to about 3000 hr·μg/mL.

Embodiment 8 provides the method of any one of embodiments 1-7, wherein the individual is human.

Embodiment 9 provides the method of any one of embodiments 1-8, wherein the composition comprises at least one additional pharmaceutically active agent.

Embodiment 10 provides the method of any one of embodiments 1-9, wherein the composition comprises at least one pharmaceutically acceptable excipient.

Embodiment 11 provides the method of any one of embodiments 1-10, wherein the composition comprises at least one pharmaceutically acceptable carrier.

Embodiment 12 provides the method of any one of embodiments 1-11, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

Embodiment 13 provides the method of any one of embodiments 1-12, wherein the composition is administered orally.

Embodiment 14 provides the method of any one of embodiments 1-13, wherein the composition is administered in a form comprising a tablet, hard capsule, soft capsule, cachet, troche, lozenge, or suppository.

Embodiment 15 provides a method of treating post-surgical pain, the method comprising administering a therapeutically effective amount of a composition comprising a compound of Formula I:

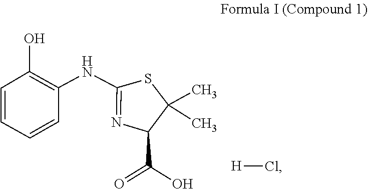 [SMILES: CC1(C)SC(Nc2ccccc2O)=N[C@@H]1C(=O)O ; Cl]

to an individual having post-surgical pain.

Embodiment 16 provides the method of embodiment 15, wherein the post-surgical pain is present at or near at least one surgical site.

Embodiment 17 provides the method of any one of embodiments 15-16, wherein the surgical site comprises at least one incision.

Embodiment 18 provides the method of any one of embodiments 15-17, wherein the at least one surgical site results from a surgery or procedure selected from the group consisting of appendectomy, arthroscopic surgery, brain surgery, breast biopsy, carotid endarterectomy, cataract surgery, Cesarean section, cholecystectomy, circumcision, coronary artery bypass, colon or rectal, debridement of wound, burn, or infection, dilation and curettage, endoscopy, free skin graft, gastric bypass, hemorrhoidectomy, hip replacement, hysterectomy, hysteroscopy, inguinal hernia repair, low back pain surgery, liver resection, lung resection, mastectomy (partial, total, or modified radical), mediport insertion or removal, orthopedic surgery, partial colectomy, parathyroidectomy, prostatectomy, spinal surgery, tubal ligation, thyroidectomy, tonsillectomy, and combinations thereof.

Embodiment 19 provides the method of any one of embodiments 15-18, wherein the therapeutically effective amount of Compound 1 comprises about 5 mg to about 5000 mg.

Embodiment 20 provides the method of any one of embodiments 15-19, wherein composition is administered for about 1 day to about 90 days.

Embodiment 21 provides the method of any one of embodiments 15-20, wherein administration of the composition results in a maximum observed plasma concentration (Cₘₐₓ) of about 5 μg/mL to about 300 μg/mL.

Embodiment 22 provides the method of any one of embodiments 15-21, wherein administration of the composition results in an area under the curve (AUC_{INF}) of about 100 hr·μg/mL to about 3000 hr·μg/mL.

Embodiment 23 provides the method of any one of embodiments 15-22, wherein the individual is human.

Embodiment 24 provides the method of any one of embodiments 15-23, wherein the composition comprises at least one additional pharmaceutically active agent.

Embodiment 25 provides the method of any one of embodiments 15-24, wherein the composition comprises at least one pharmaceutically acceptable excipient.

Embodiment 26 provides the method of any one of embodiments 15-25, wherein the composition comprises at least one pharmaceutically acceptable carrier.

Embodiment 27 provides the method of any one of embodiments 15-26, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

Embodiment 28 provides the method of any one of embodiments 15-27, wherein the composition is administered orally.

Embodiment 29 provides the method of any one of embodiments 15-28, wherein the composition is administered in a form comprising a tablet, hard capsule, soft capsule, cachet, troche, lozenge, or suppository.

Embodiment 30 provides a method of making a compound of Formula I,

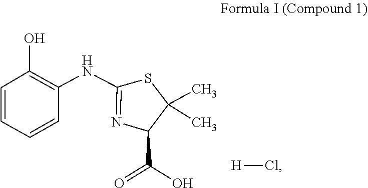 [SMILES: CC1(C)SC(Nc2ccccc2O)=N[C@@H]1C(=O)O ; Cl]

the method comprising:

reacting an amine with a structure of

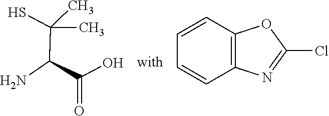

in the presence of a base and a first solvent to form an intermediate product of Formula II:

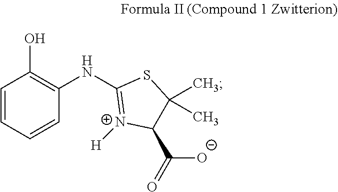 [SMILES: CC1(C)SC(Nc2ccccc2O)=[NH+][C@@H]1C(=O)[O-]]

and contacting the intermediate product with an acid and a second solvent to form the compound of Formula I.

Embodiment 31 provides the method of embodiment 30, wherein the base comprises an alkali metal hydroxide.

Embodiment 32 provides the method of any one of embodiments 30-31, wherein the alkali metal hydroxide is selected from the group consisting of LiOH, NaOH, KOH, and any combinations thereof.

Embodiment 33 provides the method of any one of embodiments 30-32, wherein the alkali metal hydroxide is NaOH.

Embodiment 34 provides the method of any one of embodiments 30-33, wherein the first solvent comprises a polar protic solvent, a polar aprotic solvent, or any combinations thereof.

Embodiment 35 provides the method of any one of embodiments 30-34, wherein the first solvent is a polar protic solvent.

Embodiment 36 provides the method of any one of embodiments 30-35, wherein the first solvent is water.

Embodiment 37 provides the method of any one of embodiments 30-36, wherein the intermediate product is isolated prior to contacting with the acid and the second solvent.

Embodiment 38 provides the method of any one of embodiments 30-37, wherein the acid is an inorganic acid or an organic acid.

Embodiment 39 provides the method of any one of embodiments 30-38, wherein the acid is an inorganic acid.

Embodiment 40 provides the method of any one of embodiments 30-39, wherein the acid is hydrochloric acid (HCl).

Embodiment 41 provides a kit comprising a composition comprising a compound of Formula I,

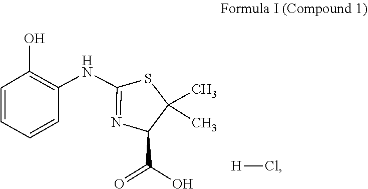 [SMILES: CC1(C)SC(Nc2ccccc2O)=N[C@@H]1C(=O)O ; Cl]

an applicator, and instructional material for use thereof, wherein the instructional material comprises instructions for treating diabetic neuropathy or post-surgical pain.

Embodiment 42 provides the kit of embodiment 41, wherein the instructional material comprises instructions for administering the composition comprising about 5 mg to about 500 mg of Compound 1.

Embodiment 43 provides the kit of any one of embodiments 41-42, wherein the instructional material comprises instructions for treating diabetic neuropathy.

Embodiment 44 provides the kit of any one of embodiments 41-43, wherein the instructional material comprises instructions for treating post-surgical pain.

1. A method of treating diabetic neuropathy, the method comprising administering a therapeutically effective amount of a composition comprising a compound of Formula I: 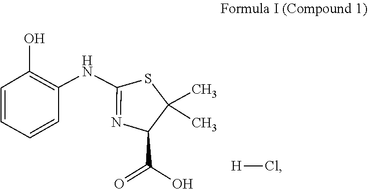 [SMILES: CC1(C)SC(Nc2ccccc2O)=N[C@@H]1C(=O)O ; Cl]
to an individual having diabetic neuropathy.

2. The method of claim 1, wherein the diabetic neuropathy comprises peripheral neuropathy, proximal neuropathy, autonomic neuropathy, focal neuropathy, or combinations thereof.

3. The method of claim 1, wherein the individual has type I or type II diabetes.

4. The method of claim 1, wherein the therapeutically effective amount of Compound 1 comprises about 5 mg to about 5000 mg.

5. The method of claim 1, wherein the composition is administered for about 1 day to about 90 days.

6. The method of claim 1, wherein administration of the composition results in a maximum observed Compound I plasma concentration (Cₘₐₓ) of about 5 μg/mL to about 300 μg/mL.

7. The method of claim 1, wherein administration of the composition results in an area under the curve (AUC_{INF}) of about 100 hr·μg/mL to about 3000 hr·μg/mL for Compound I.

8. The method of claim 1, wherein the individual is human.

9. The method of claim 1, wherein the composition comprises at least one additional pharmaceutically active agent, pharmaceutically acceptable excipient, or pharmaceutically acceptable carrier.

10. The method of claim 1, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

11. The method of claim 10, wherein the composition is administered in a form comprising a tablet, hard capsule, soft capsule, cachet, troche, lozenge, or suppository.

12. A method of treating post-surgical pain, the method comprising administering a therapeutically effective amount of a composition comprising a compound of Formula I: 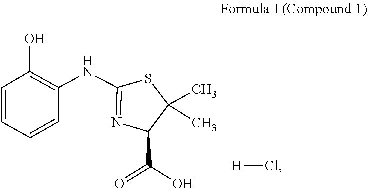 [SMILES: CC1(C)SC(Nc2ccccc2O)=N[C@@H]1C(=O)O ; Cl]
to an individual having post-surgical pain.

13. The method of claim 12, wherein the post-surgical pain is present at or near at least one surgical site.

14. The method of claim 13, wherein the surgical site comprises at least one incision.

15. The method of claim 13, wherein the at least one surgical site results from a surgery or procedure selected from the group consisting of appendectomy, arthroscopic surgery, brain surgery, breast biopsy, carotid endarterectomy, cataract surgery, Cesarean section, cholecystectomy, circumcision, coronary artery bypass, colon or rectal, debridement of wound, burn, or infection, dilation and curettage, endoscopy, free skin graft, gastric bypass, hemorrhoidectomy, hip replacement, hysterectomy, hysteroscopy, inguinal hernia repair, low back pain surgery, liver resection, lung resection, mastectomy (partial, total, or modified radical), mediport insertion or removal, orthopedic surgery, partial colectomy, parathyroidectomy, prostatectomy, spinal surgery, tubal ligation, thyroidectomy, tonsillectomy, and combinations thereof.

16. The method of claim 12, wherein the therapeutically effective amount of Compound 1 comprises about 5 mg to about 5000 mg.

17. The method of claim 12, wherein the composition is administered for about 1 day to about 90 days.

18. The method of claim 12, wherein administration of the composition results in a maximum observed Compound I plasma concentration (Cₘₐₓ) of about 5 μg/mL to about 300 μg/mL.

19. The method of claim 12, wherein administration of the composition results in an area under the curve (AUC_{INF}) of about 100 hr·μg/mL to about 3000 hr·μg/mL for Compound I.

20. The method of claim 12, wherein the individual is human.

21. The method of claim 12, wherein the composition comprises at least one additional pharmaceutically active agent, one pharmaceutically acceptable excipient, or pharmaceutically acceptable carrier.

22. The method of claim 12, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

23. The method of claim 22, wherein the composition is administered in a form comprising a tablet, hard capsule, soft capsule, cachet, troche, lozenge, or suppository.

24. A method of making a compound of Formula I, 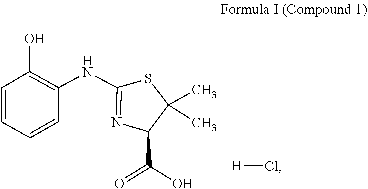 [SMILES: CC1(C)SC(Nc2ccccc2O)=N[C@@H]1C(=O)O ; Cl]
the method comprising:
reacting an amine with a structure of 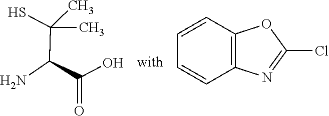
in the presence of a base and a first solvent to form an intermediate product of Formula II: 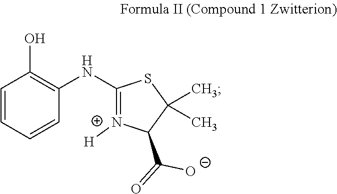 [SMILES: CC1(C)SC(Nc2ccccc2O)=[NH+][C@@H]1C(=O)[O-]]
and contacting the intermediate product with an acid and a second solvent to form the compound of Formula I.

25. The method of claim 24, wherein the base comprises an alkali metal hydroxide.

26. The method of claim 25, wherein the alkali metal hydroxide is selected from the group consisting of LiOH, NaOH, KOH, and combinations thereof.

27. The method of claim 25, wherein the first solvent comprises a polar protic solvent, a polar aprotic solvent, or any combinations thereof.

28. The method of claim 25, wherein the intermediate product is isolated prior to contacting with the acid and the second solvent.

29. The method of claim 25, wherein the acid is an inorganic acid or an organic acid.

30. A kit comprising a composition comprising a compound of Formula I: 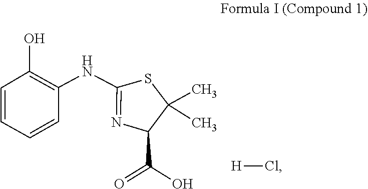 [SMILES: CC1(C)SC(Nc2ccccc2O)=N[C@@H]1C(=O)O ; Cl]
an applicator, and instructional material for use thereof, wherein the instructional material comprises instructions for treating diabetic neuropathy or post-surgical pain.

31. The method of claim 26, wherein the alkali metal hydroxide is NaOH.

32. The method of claim 27, wherein the first solvent is water.

33. The method of claim 28, wherein the second solvent is iso-propyl alcohol.

34. The method of claim 29, wherein the acid is HCl.
